(19) United States
(12) Patent Application Publication
(10) Pub. No.: US 2023/0181878 A1
(43) Pub. Date: Jun. 15, 2023

(54) Sealing Modules for Insertion Assemblies of Rapidly Insertable Central Catheters and Methods Thereof

(71) Applicant: Bard Access Systems, Inc., Salt Lake City, UT (US)

(72) Inventors: Daniel B. Blanchard, Bountiful, UT (US); Glade H. Howell, Draper, UT (US); Kyle G. Thornley, Farmington, UT (US); Eric W. Lindekugel, Salt Lake City, UT (US)

(21) Appl. No.: 18/081,491

(22) Filed: Dec. 14, 2022

Related U.S. Application Data
(60) Provisional application No. 63/290,049, filed on Dec. 15, 2021.

(51) Int. Cl. A61M 25/09; A61M 25/06; A61M 25/00

(52) U.S. Cl. CPC A61M 25/09; A61M 25/0606; A61M 25/0043

(57) ABSTRACT

A rapidly insertable central catheter (“RICC”) insertion assembly having a sealing module can include a RICC, an introducer needle, an access guidewire, and a coupler coupling the RICC and the introducer needle together. A proximal portion of the access guidewire is disposed in a primary lumen of the RICC. A distal portion of the access guidewire is disposed in a needle shaft of the introducer needle through a needle slot thereof. A coupler housing of the coupler includes a sealing-module cavity of the sealing module. An elastomeric sealing-module insert of the sealing module is inserted into the sealing-module cavity. The sealing-module insert is configured to separately seal around a proximal portion of the introducer needle and the distal portion of the access guidewire when the sealing-module insert is compressed in the sealing-module cavity. A method can include inserting the foregoing RICC into a blood-vessel lumen of a patient.

### PRIORITY

This application claims the benefit of priority to U.S. Provisional Application No. 63/290,049, filed Dec. 15, 2021, which is incorporated by reference in its entirety into this application.

### BACKGROUND

Central venous catheters (“CVCs”) are commonly introduced into patients and advanced through their vasculatures by way of the Seldinger technique. The Seldinger technique utilizes a number of steps and medical devices (e.g., a needle, a scalpel, a guidewire, an introducer sheath, a dilator, a CVC, etc.). While the Seldinger technique is effective, the number of steps are time consuming, handling the number of medical devices is awkward, and both of the foregoing can lead to patient trauma. In addition, there is a relatively high potential for touch contamination due to the number of medical devices that need to be interchanged during the Seldinger technique. As such, there is a need to reduce the number of steps and medical devices involved in introducing a catheter such as a CVC into a patient and advancing the catheter through a vasculature thereof.

Disclosed herein are insertion assemblies of rapidly insertable central catheters (“RICCs”) and methods that address the foregoing. Notably, the RICC insertion assemblies disclosed herein include sealing modules for sealing different components of the RICC insertion assemblies therein.

### SUMMARY

Disclosed herein is a RICC insertion assembly including, in some embodiments, a RICC, an introducer needle, an access guidewire, and a coupler coupling the RICC and the introducer needle together. The introducer needle includes a needle shaft having a longitudinal needle slot. The access guidewire has a proximal portion and a distal portion. The proximal portion of the access guidewire is disposed in a primary lumen of the RICC. The distal portion of the access guidewire is disposed in the needle shaft through the needle slot. The coupler includes a coupler housing. The coupler housing includes a sealing-module cavity of a sealing module. An elastomeric sealing-module insert of the sealing module is inserted into the sealing-module cavity. The sealing-module insert is configured to separately seal around a proximal portion of the introducer needle and the distal portion of the access guidewire when the sealing-module insert is compressed in the sealing-module cavity in one or more states of the RICC insertion assembly.

In some embodiments, the sealing-module insert includes an introducer-needle passageway and an access-guidewire passageway. The introducer-needle passageway is configured to seal around the proximal portion of the introducer needle when the sealing-module insert is compressed in the sealing-module cavity. In addition, the access-guidewire passageway is configured to seal around the distal portion of the access guidewire when the sealing-module insert is compressed in the sealing-module cavity.

In some embodiments, the introducer-needle passageway includes an internal relief between proximal and distal portions of the introducer-needle passageway. With the internal relief between proximal and distal portions of the introducer-needle passageway, only proximal- and distal-end portions of the introducer-needle passageway are configured to seal around the proximal portion of the introducer needle when the sealing-module insert is compressed in the sealing-module cavity.

In some embodiments, the access-guidewire passageway includes an internal relief in at least a proximal portion of the access-guidewire passageway. With the internal relief in the proximal portion of the access-guidewire passageway, only a proximal-end portion of the access-guidewire passageway is configured to seal around the distal portion of the access guidewire when the sealing-module insert is compressed in the sealing-module cavity.

In some embodiments, a distal end of the access-guidewire passageway connects to a medial portion of the introducer-needle passageway. Connecting the access-guidewire passageway to the introducer-needle passageway allows the distal portion of the access guidewire to be disposed in the needle shaft through the needle slot.

In some embodiments, the sealing-module insert includes a partial-length longitudinal slit between the access-guidewire passageway and the introducer-needle passageway. The partial-length longitudinal slit is configured to allow the access guidewire to escape from the access-guidewire passageway into the introducer-needle passageway after the introducer needle is withdrawn from the RICC insertion assembly through the introducer-needle passageway.

In some embodiments, the sealing-module insert includes a full-length longitudinal slit between the introducer-needle passageway and an exterior of the sealing-module insert. The full-length longitudinal slit is configured to allow the access guidewire to escape from the introducer-needle passageway after the introducer needle is withdrawn from the RICC insertion assembly through the introducer-needle passageway.

In some embodiments, the full-length longitudinal slit in the sealing-module insert opens into a longitudinal coupler-housing slot of the coupler housing. The coupler-housing slot is configured to allow the access guidewire to escape from the coupler housing after the introducer needle is withdrawn from the RICC insertion assembly through the introducer-needle passageway.

In some embodiments, the sealing-module insert includes an external relief between the sealing-module insert and the coupler housing coextensive with the full-length longitudinal slit in the sealing-module insert. The external relief is configured to allow the access guidewire to escape from the introducer-needle passageway without binding the access guidewire in the full-length longitudinal slit.

In some embodiments, the sealing-module insert is radially compressed in the sealing-module cavity.

In some embodiments, the sealing-module insert is axially compressed in the sealing-module cavity by a follower proximal of the sealing-module insert. Axial compression of the sealing-module insert in the sealing-module cavity, in turn, radially compresses the sealing-module insert in the sealing-module cavity.

In some embodiments, the follower is a distal portion of a needle hub of the introducer needle removably disposed in a needle-hub receptacle of the coupler housing. The needle hub has a proximal portion including a Luer connector for fluidly connecting a syringe to the introducer needle.

In some embodiments, the coupler includes a needle-hub lock configured to lock the needle hub in the needle-hub receptacle. A pair of lock buttons of the needle-hub lock is distributed between opposite sides of the coupler. The pair of lock buttons is configured to unlock the needle hub when the lock buttons are pressed into the coupler for withdrawal of the introducer needle from the coupler through the introducer-needle passageway.

In some embodiments, the introducer needle further includes a sheath over the needle shaft sealing the needle slot thereunder. The needle slot extends from a proximal portion of the needle shaft through a distal needle tip.

In some embodiments, the sealing module includes a blade disposed in the needle slot under a distal end of a sheath opening. The blade includes a distal-facing blade edge configured to cut the sheath away from the needle shaft as the introducer needle is withdrawn from the coupler through the introducer-needle passageway. Cutting the sheath away from the needle shaft as the introducer needle is withdrawn from the coupler allows the access guidewire to escape from the needle shaft by way of the needle slot.

In some embodiments, the blade is overmolded into the sealing-module insert, thereby integrating the blade in the sealing-module insert.

In some embodiments, the blade extends from a blade holder of a blade module. The blade module is disposed in a sealing module-insert cavity of the sealing-module insert.

In some embodiments, a proximal end of the access guidewire is coupled to a swivel arm swivelably coupled to the coupler housing. Coupling the proximal end of the access guidewire to the swivel arm enforces a loop in the access guidewire with the distal portion of the access guidewire disposed in the needle shaft. The RICC is disposed over the loop in at least a ready-to-operate state of the RICC insertion assembly.

Also disclosed herein is a RICC insertion assembly including, in some embodiments, a RICC, an introducer needle, a coupler coupling the RICC and the introducer needle together, and an access guidewire disposed in both the RICC and the introducer needle in at least a ready-to-operate state of the RICC insertion assembly. The RICC includes a catheter tube, a catheter hub coupled to a proximal portion of the catheter tube, one or more extension legs, and one or more extension-leg connectors. Each extension leg of the one-or-more extension legs is coupled to the catheter hub by a distal portion thereof. Each extension-leg connector of the one-or-more extension-leg connectors is over a proximal portion of an extension leg of the one-or-more extension legs. The introducer needle includes a needle shaft, a sheath over the needle shaft, and a needle hub over a proximal portion of the needle shaft and a proximal portion of the sheath. The needle shaft includes a longitudinal needle slot extending from the proximal portion of the needle shaft through a distal needle tip. The sheath includes a sheath opening in the proximal portion of the sheath. The coupler includes a coupler housing and an extension arm coupled to the coupler housing. The coupler housing includes a sealing-module cavity of a sealing module and a needle-hub receptacle proximal of the sealing-module cavity. An elastomeric sealing-module insert of the sealing module is inserted into the sealing-module cavity, and the needle hub is inserted into the needle-hub receptacle. The extension arm includes an extension-arm connector connected to an extension-leg connector of the one-or-more extension-leg connectors. The access guidewire includes a proximal portion with a proximal end and a distal portion with a distal end. The proximal end of the access guidewire is coupled to the extension-arm connector. The proximal portion of the access guidewire extends along a primary lumen of the RICC. The distal portion of the access guidewire extends along the primary lumen of the RICC, out a distal end of the RICC, into the sealing module over the needle hub, into the needle shaft through both the sheath opening and the needle slot, and along a needle lumen of the introducer needle. The distal end of the access guidewire is disposed in the needle lumen just proximal of the needle tip, thereby enforcing a loop in the access guidewire over which loop the RICC is disposed. The sealing-module insert separately seals around both a proximal portion of the introducer needle and the distal portion of the access guidewire with the sealing-module insert compressed in the sealing-module cavity by the needle hub.

Also disclosed herein is a method for inserting a RICC into a blood-vessel lumen of a patient. The method includes, in some embodiments, an insertion assembly-obtaining step, a needle tract-establishing step, an access guidewire-advancing step, and a RICC-advancing step. The insertion assembly-obtaining step includes obtaining a RICC insertion assembly, optionally, already in a ready-to-operate state thereof. The RICC insertion assembly includes the RICC, an introducer needle, an access guidewire, and a coupler coupling the RICC and the introducer needle together. A sealing module of the coupler formed between at least a sealing-module cavity of a coupler housing and an elastomeric sealing-module insert disposed in the sealing-module cavity separately seals around a proximal portion of the introducer needle and a distal portion of the access guidewire in at least the ready-to-operate state of the RICC insertion assembly. The needle tract-establishing step includes establishing a needle tract from an area of skin to the blood-vessel lumen with the introducer needle. The access guidewire-advancing step includes advancing the distal portion of the access guidewire through both the sealing module and a longitudinal needle slot of a needle shaft of the introducer needle. In the access guidewire-advancing step, a distal end of the access guidewire is advanced from a location in the introducer needle just proximal of a needle tip of the needle shaft into the blood-vessel lumen. The RICC-advancing step includes advancing a catheter tube of the RICC over the access guidewire and into the blood-vessel lumen, thereby inserting the RICC into the blood-vessel lumen.

In some embodiments, a distal end of an access-guidewire passageway in the sealing-module insert connects to a medial portion of an introducer-needle passageway in the sealing-module insert. Connecting the distal end of an access-guidewire passageway to the medial portion of an introducer-needle passageway allows the distal portion of the access guidewire to be disposed in the needle shaft through the needle slot of the needle shaft.

In some embodiments, the method further includes an introducer needle-withdrawing step. The introducer needle-withdrawing step includes withdrawing the introducer needle from the coupler leaving the access guidewire in place in the blood-vessel lumen. The introducer needle-withdrawing step removes a needle hub of the introducer needle from a needle-hub receptacle of the coupler housing, thereby removing both axial and radial compression on the sealing-module insert and unsealing the proximal portion of the introducer needle and the distal portion of the access guidewire. The introducer needle-withdrawing step is performed before the RICC-advancing step of advancing the catheter tube over the access guidewire and into the blood-vessel lumen.

In some embodiments, the method further includes an unlocking step. The unlocking step includes pressing a pair of lock buttons of a needle-hub lock distributed between opposite sides of the coupler into the coupler to unlock the needle hub. The unlocking step is performed before the introducer needle-withdrawing step of withdrawing the introducer needle from the coupler.

In some embodiments, the introducer needle-withdrawing step of withdrawing the introducer needle from the coupler includes cutting a needle slot-sealing sheath of the introducer needle away from the needle shaft. The sealing module includes a blade disposed in the needle slot under a distal end of a sheath opening of the sheath with a distal-facing blade edge for the cutting of the sheath away from the needle shaft.

In some embodiments, the cutting of the sheath away from the needle shaft allows the access guidewire to escape from the needle shaft by way of the needle slot.

In some embodiments, a longitudinal coupler-housing slot of the coupler housing allows the access guidewire to escape from the coupler housing after the introducer needle-withdrawing step of withdrawing the introducer needle from the coupler.

In some embodiments, the needle tract-establishing step includes ensuring blood flashes back into the introducer needle or a syringe fluidly connected to the introducer needle, thereby confirming the needle tract extends into the blood-vessel lumen.

In some embodiments, the method further includes a blood-aspirating step. The blood-aspirating step includes aspirating blood with the syringe to confirm the needle tract extends into the blood-vessel lumen before the introducer needle-withdrawing step of withdrawing the introducer needle from the coupler.

In some embodiments, the method further includes an access guidewire-withdrawing step. The access guidewire-withdrawing step includes withdrawing the access guidewire leaving the catheter tube in place in the blood-vessel lumen.

In some embodiments, the method further includes a maneuver guidewire-advancing step, another RICC-advancing step, and a maneuver guidewire-withdrawing step. The maneuver guidewire-advancing step includes advancing a maneuver guidewire into the blood-vessel lumen by way of a primary lumen of the RICC. The other RICC-advancing step includes advancing a distal portion of the catheter tube farther into the blood-vessel lumen over the maneuver guidewire to a lower ⅓ of a superior vena cava (“SVC”) of a heart of the patient. The maneuver guidewire-withdrawing step includes withdrawing the maneuver guidewire leaving the catheter tube in place in the lower ⅓ of the SVC.

These and other features of the concepts provided herein will become more apparent to those of skill in the art in view of the accompanying drawings and following description, which describe particular embodiments of such concepts in greater detail.

### DRAWINGS

FIG. 1 illustrates a top view of a RICC insertion assembly in accordance with some embodiments.

FIG. 2 illustrates a perspective view of the RICC insertion assembly in accordance with some embodiments.

FIG. 3 illustrates a bottom view of the RICC insertion assembly in accordance with some embodiments.

FIG. 4 illustrates a cutaway view of a coupler of the RICC insertion assembly in accordance with some embodiments.

FIG. 5 illustrates another cutaway view of the coupler in accordance with some embodiments.

FIG. 6 illustrates yet another cutaway view of the coupler in accordance with some embodiments.

FIG. 7 illustrates a longitudinal cross section of the coupler, a sealing module of the coupler, and an introducer needle of the RICC insertion assembly in accordance with some embodiments.

FIG. 8 illustrates a longitudinal cross section of the coupler, the sealing module of the coupler, the introducer needle, and an access guidewire of the RICC insertion assembly in accordance with some embodiments.

FIG. 9 illustrates a perspective view of a proximal end of a sealing-module insert of the sealing module in accordance with some embodiments.

FIG. 10 illustrates a perspective view of a distal end of the sealing-module insert in accordance with some embodiments.

FIG. 11 illustrates longitudinal cross section of the sealing-module insert in accordance with some embodiments.

FIG. 12 illustrates radial compression of the sealing-module insert in a sealing-module cavity of a coupler housing of the coupler in accordance with some embodiments.

FIG. 13 illustrates radial compression of the sealing-module insert in the sealing-module cavity through axial compression of the sealing-module insert in the sealing-module cavity by a needle hub in accordance with some embodiments.

FIG. 14 illustrates a longitudinal cross section of another sealing-module insert with internal reliefs in an introducer-needle passageway and an access-guidewire passageway of the sealing-module insert in accordance with some embodiments.

FIG. 15 illustrates a transverse cross section of a distal portion of the sealing module in accordance with some embodiments.

FIG. 16 illustrates a transverse cross section of the distal portion of the sealing module with an external relief between the sealing-module insert and the coupler housing in accordance with some embodiments.

FIG. 17 illustrates a transverse cross section of a distal portion of the coupler and the sealing module thereof with the external relief between the sealing-module insert and the coupler housing in accordance with some embodiments.

FIG. 18 illustrates the sealing-module insert having a first shape in accordance with some embodiments.

FIG. 19 illustrates the sealing-module insert having a second shape in accordance with some embodiments.

FIG. 20 illustrates the sealing-module insert having a third shape in accordance with some embodiments.

FIG. 21 illustrates the sealing-module insert having a fourth shape disposed in the sealing-module cavity of the coupler housing in accordance with some embodiments

FIG. 22 illustrates a top view of the introducer needle in accordance with some embodiments.

FIG. 23 illustrates a sheath of the introducer needle in accordance with some embodiments.

FIG. 24 illustrates a needle shaft of the introducer needle in accordance with some embodiments.

FIG. 25 illustrates a RICC of the RICC insertion assembly in accordance with some embodiments.

FIG. 26 illustrates a detailed view of a distal portion of a catheter tube of the RICC in accordance with some embodiments.

FIG. 27 illustrates a transverse cross section of the distal portion of the catheter tube in accordance with some embodiments.

FIG. 28 illustrates another transverse cross section of the distal portion of the catheter tube in accordance with some embodiments.

FIG. 29 illustrates a longitudinal cross section of the distal portion of the catheter tube in accordance with some embodiments.

FIG. 30 illustrates a needle tract-establishing step of a method of using the RICC insertion assembly in accordance with some embodiments.

FIG. 31 illustrates a blood-aspirating step of the method in accordance with some embodiments.

FIG. 32 illustrates an access guidewire-advancing step of the method in accordance with some embodiments.

FIG. 33 illustrates an introducer needle-withdrawing step of the method in accordance with some embodiments.

FIG. 34 illustrates a RICC-advancing step of the method in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

As set forth above with respect to the Seldinger technique, the number of steps are time consuming, handling the number of medical devices is awkward, and both of the foregoing can lead to patient trauma. In addition, there is a relatively high potential for touch contamination due to the number of medical devices that need to be interchanged during the Seldinger technique. As such, there is a need to reduce the number of steps and medical devices involved in introducing a catheter such as a CVC into a patient and advancing the catheter through a vasculature thereof.

Disclosed herein are insertion assemblies of RICCs and methods that address the foregoing. Notably, the RICC insertion assemblies disclosed herein include sealing modules for sealing different components of the RICC insertion assemblies therein. For example, a RICC insertion assembly can include a RICC, an introducer needle, an access guidewire, and a coupler coupling the RICC and the introducer needle together. A proximal portion of the access guidewire is disposed in a primary lumen of the RICC. A distal portion of the access guidewire is disposed in a needle shaft of the introducer needle through a needle slot thereof. A coupler housing of the coupler includes a sealing-module cavity of a sealing module. An elastomeric sealing-module insert of the sealing module is inserted into the sealing-module cavity. The sealing-module insert is configured to separately seal around a proximal portion of the introducer needle and the distal portion of the access guidewire when the sealing-module insert is compressed in the sealing-module cavity.

The foregoing features as well as other features of the RICC insertion assemblies and methods disclosed herein will become more apparent to those of skill in the art in view of the accompanying drawings and following description, which describe particular embodiments of the RICC insertion assemblies and methods in greater detail. However, it should be understood the RICCs of the RICC insertion assemblies are but one type of catheter that can be incorporated into catheter insertion assemblies like those disclosed herein. Indeed, peripherally inserted central catheters (“PICCs”), dialysis catheters, or the like can also be incorporated into catheter insertion assemblies and methods.

### RICC Insertion Assemblies

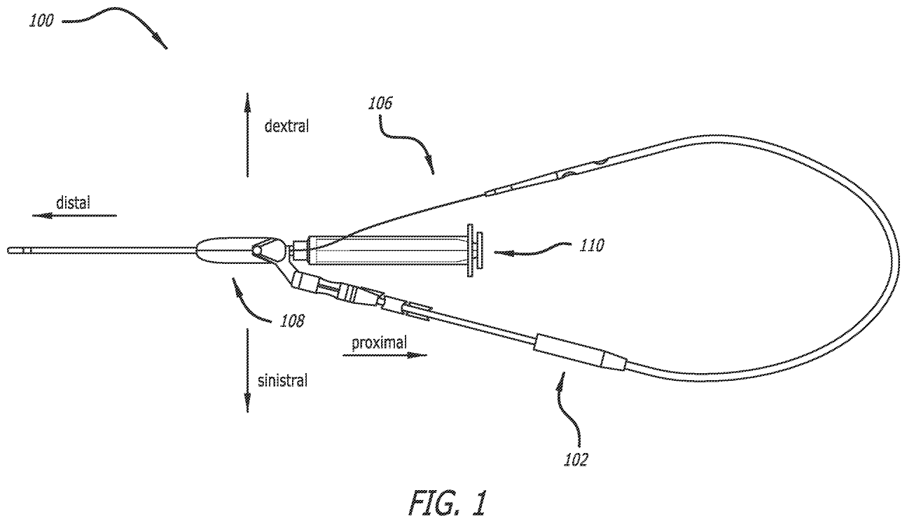

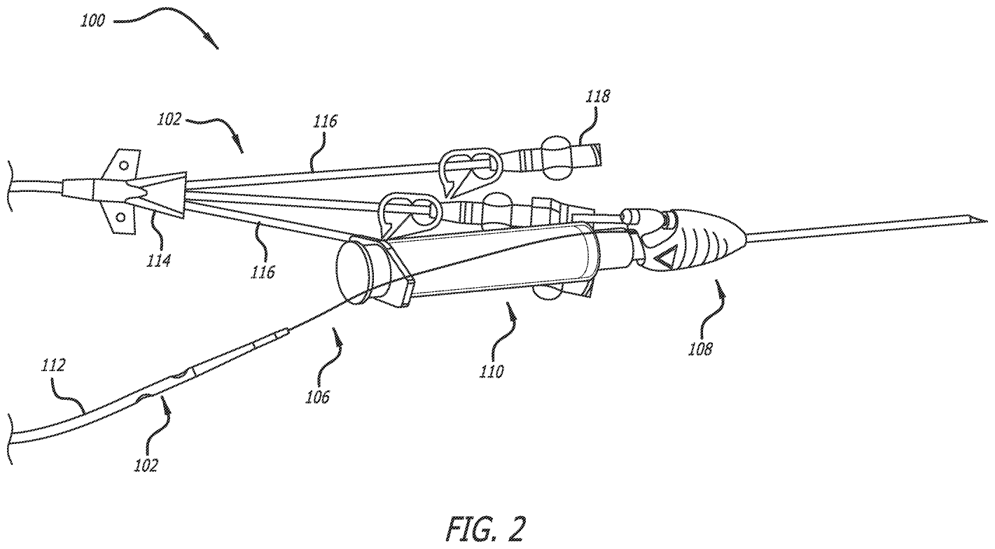

FIGS. 1-3 illustrate various views of a RICC insertion assembly 100 in accordance with some embodiments.

As shown, the RICC insertion assembly 100 includes a RICC 102, an introducer needle 104, an access guidewire 106, and a coupler 108 coupling the RICC 102, the introducer needle 104, and the access guidewire 106 together in a ready-to-operate state of the RICC insertion assembly 100. Notably, the proximal end of the access guidewire 106 is coupled to the coupler 108 and the distal end of the access guidewire 106 is disposed in the needle lumen 158 of the introducer needle 104 as set forth below. This enforces a loop in the access guidewire 106, which loop the RICC 102 is disposed over in the ready-to-operate state of the RICC insertion assembly 100 keeping the RICC insertion assembly 100 in a relatively compact form.

The RICC insertion assembly 100 can further include a syringe 110 fluidly coupled to the introducer needle 104 in the ready-to-operate state of the RICC insertion assembly 100. As set forth below, the sheath 142 seals the needle slot 148 of the needle shaft 140. In particular, the sheath 142 seals the needle slot 148 outside of the sealing module 180. The sealing module 180, in turn, seals over the sheath opening 162 of the sheath 142 that opens to the needle slot 148. The sealing module 180 also seals around the proximal portion of the introducer needle 104 and the distal portion of the access guidewire 106 when the sealing-module insert 202 is compressed in the sealing-module cavity 198 in one or more states of the RICC insertion assembly 100. Such seals enable the syringe 110 to aspirate blood in accordance with the blood-aspirating step of the method set forth below.

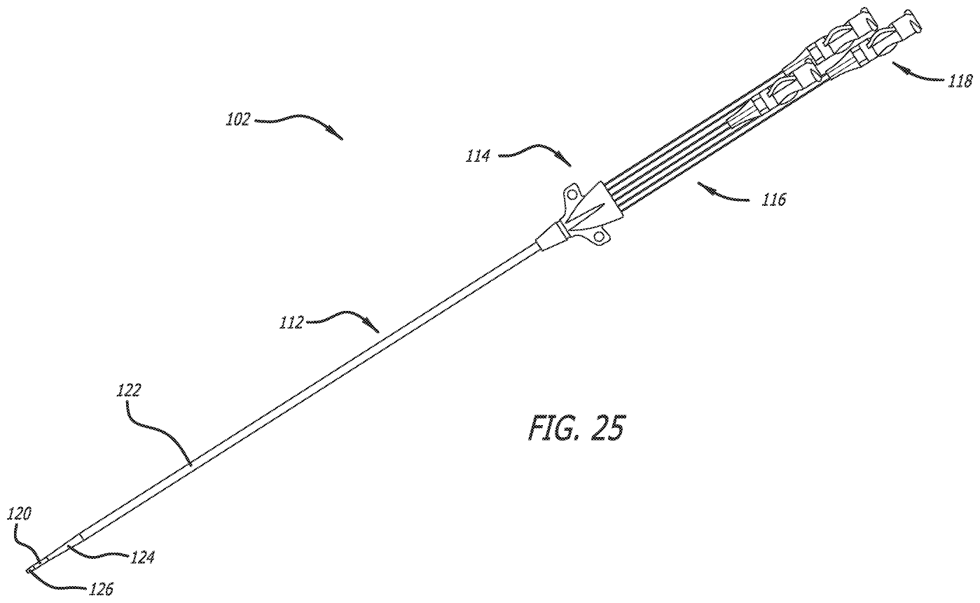

FIG. 25 illustrates the RICC 102 of the RICC insertion assembly 100 in accordance with some embodiments.

As shown, the RICC 102 includes a catheter tube 112, a catheter hub 114, one or more extension legs 116, and one or more extension-leg connectors 118.

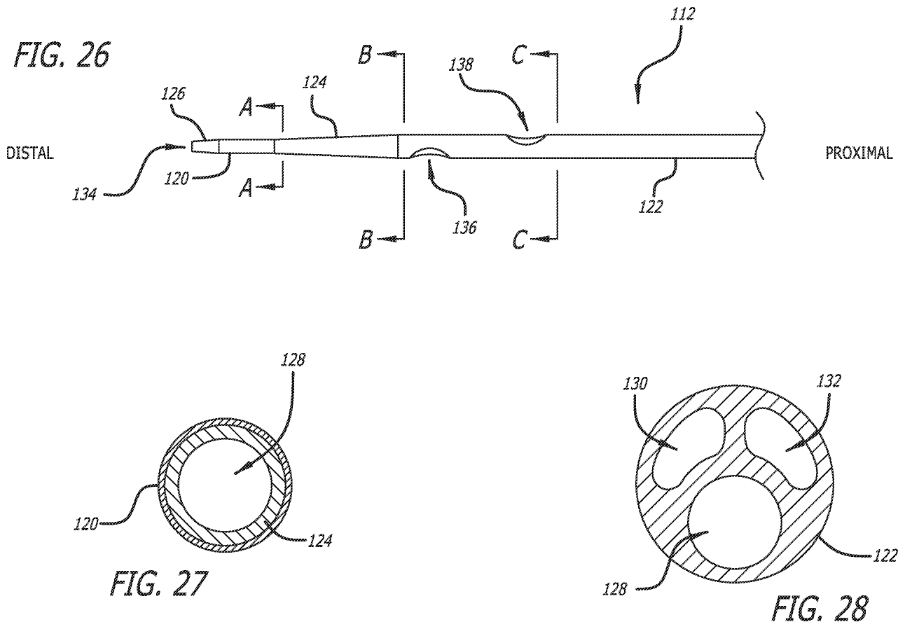

FIGS. 26-29 illustrate various views of the catheter tube 112 of the RICC 102 in accordance with some embodiments.

The catheter tube 112 includes a first section 120 in a distal portion of the catheter tube 112, a second section 122 in the distal portion of the catheter tube 112 proximal of the first section 120, and a tapered junction 124 between the first and second sections 120 and 122 of the catheter tube 112.

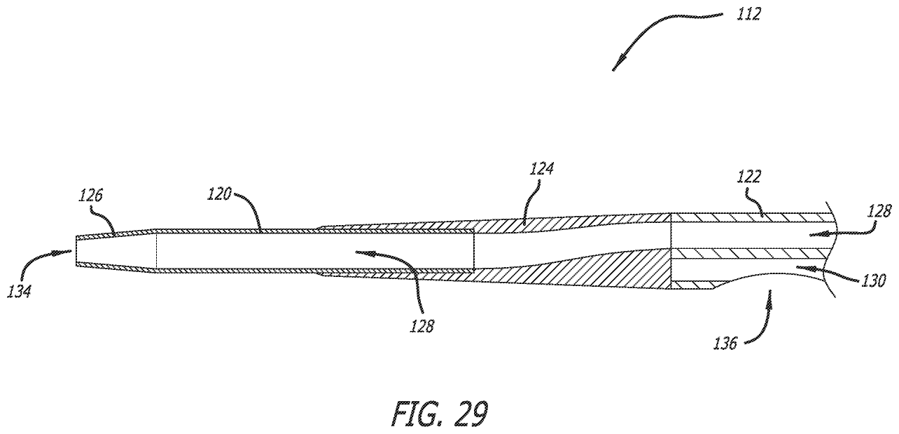

The first section 120 of the catheter tube 112 includes a catheter tip 126 having a relatively short taper from an outer diameter of a distal portion of the first section 120 distal of the junction 124 to an outer diameter of a distal end of the first section 120. The taper of the catheter tip 126 is configured for immediate dilation of tissue about a needle tract established with the introducer needle 104 up to the outer diameter of the distal portion of the first section 120 of the catheter tube 112. As best shown in FIG. 29, the first section 120 of the catheter tube 112 also includes a proximal portion disposed in a bore of a distal portion of the junction 124 and fixedly coupled thereto such as by a solvent bond, an adhesive bond, or a heat weld.

The second section 122 of the catheter tube 112 includes a consistent outer diameter over its length from a distal end of the second section 122 to a proximal end of the second section 122. The consistent diameter of the second section 122 of the catheter tube 112 is configured for smooth insertion into the needle tract and targeted vasculature subsequent to any dilation by the first section 120 of the catheter tube 112 and the junction 124. The distal end of the second section 122 of the catheter tube 112 has a flat face flush with the flat-faced proximal end of the junction 124 and fixedly coupled thereto such as by a solvent bond, an adhesive bond, or a heat weld.

The junction 124 includes a taper over its length from a proximal end of the junction 124 to a distal end of the junction 124. The taper of the junction 124 is configured for immediate dilation of the tissue about the needle tract from the outer diameter of the proximal portion of the first section 120 of the catheter tube 112 to the outer diameter of the second section 122 of the catheter tube 112. An abluminal surface of the junction 124 smoothly transitions from an abluminal surface of the first section 120 of the catheter tube 112 to an abluminal surface of the second section 122 of the catheter tube 112 without edges that catch on skin when the catheter tube 112 is inserted into the needle tract. In addition to the edges being minimal to negligible, the edges can include solvent-interdiffused polymeric material of the polymeric materials from which the catheter tube 112 is formed, which smoothens the transitions from the first section 120 of the catheter tube 112 to the junction 124 and from the junction 124 to the second section 122 of the catheter tube 112. Notably, the junction 124 has a length approximately commensurate with a length of an exposed portion of the first section 120 of the catheter tube 112 or between lengths of exposed portions of the first and second sections 120 and 122 of the catheter tube 112. As such, the length of the exposed portion of the first section 120 of the catheter tube 112 is less than the length of the junction 124 up to approximately commensurate with the length of the junction 124.

The first section 120 of the catheter tube 112 is formed of a first polymeric material (e.g., a polytetrafluoroethylene, a polypropylene, or a polyurethane) having a first durometer. The second section 122 of the catheter tube 112 is formed of a second polymeric material (e.g., a polyvinyl chloride, a polyethylene, another polyurethane, or a silicone) having a second durometer less than the first durometer. For example, the first section 120 of the catheter tube 112 can be formed of a first polyurethane having the first durometer while the second section 122 of the catheter tube 112 can be formed of a second, different polyurethane (e.g., a same or different diisocyanate or triisocyanate reacted with a different diol or triol, a different diisocyanate or triisocyanate reacted with a same or different diol or triol, a same diisocyanate or triisocyanate reacted with a same diol or triol under different conditions or with different additives, etc.) having the second durometer less than the first durometer. Indeed, polyurethanes are advantageous for the catheter tube 112 in that polyurethanes can be relatively rigid at room-temperature but become more flexible in vivo at body temperature, which reduces irritation to vessel walls as well as phlebitis. Polyurethanes are also advantageous in that they can be less thrombogenic than some other polymers. The junction 124 is formed of the second polymeric material or a third polymeric material (e.g., yet another polyurethane) having a third durometer less than the first durometer and greater than, approximately equal to, or less than the second durometer.

It should be understood the first durometer of the first polymeric material, the second durometer of the second polymeric material, and the third durometer of the third polymeric material can be on different scales (e.g., Type A or Type D). With this understanding, the second durometer of the second polymeric material or the third durometer of the third polymeric material might not be numerically less than the first durometer of the first polymeric material when the second durometer or the third durometer is less than the first durometer. Indeed, the hardness of the second polymeric material or the third polymeric material can still be less than the hardness of the first polymeric material as the different scales—each of which ranges from 0 to 100—are designed for characterizing different materials in groups of the materials having a like hardness.

In accordance with the first section 120 of the catheter tube 112, the second section 122 of the catheter tube 112, and the junction 124 between the first and second sections 120 and 122 of the catheter tube 112 set forth above, the catheter tube 112 possesses a column strength sufficient to prevent buckling of the catheter tube 112 when inserted into a needle tract established by with the introducer needle 104. The column strength of the catheter tube 112, optionally, in combination with the access guidewire 106, is also sufficient to prevent buckling of the catheter tube 112 when advanced through a vasculature of a patient without dilation of tissue about the needle tract or any blood vessels of the vasculature beforehand with a separate dilator.

It should be understood that additional configurations of the catheter tube 112 and the catheter tip 126 thereof are contemplated including a single-material catheter tube, a tapered catheter tube having a single taper from an outer diameter of a medial portion of the catheter tube 112 or even the proximal portion of the catheter tube 112 to an inner diameter of the catheter tube 112 at the catheter tip 126, a tapered catheter tip different from that disclosed herein, or even a flat catheter tip.

The catheter tube 112 includes one or more catheter-tube lumens extending through the catheter tube 112; however, only one catheter-tube lumen typically extends from a proximal end of the catheter tube 112 to a distal end of the catheter tube 112 in a multiluminal RICC (e.g., a diluminal RICC, a triluminal RICC, a tetraluminal RICC, a pentaluminal RICC, a hexaluminal RICC, etc.). (See FIGS. 26-29.) Indeed, the first section 120 of the catheter tube 112 typically includes a single lumen therethrough as shown in FIGS. 27 and 29.

The catheter hub 114 is coupled to a proximal portion of the catheter tube 112. The catheter hub 114 includes one or more catheter-hub lumens corresponding in number to the one-or-more catheter-tube lumens. The one-or-more catheter-hub lumens extends through an entirety of the catheter hub 114 from a proximal end of the catheter hub 114 to a distal end of the catheter hub 114.

Each extension leg of the one-or-more extension legs 116 is coupled to the catheter hub 114 by a distal portion thereof. The one-or-more extension legs 116 respectively include one or more extension-leg lumens, which, in turn, correspond in number to the one-or-more catheter-hub lumens. Each extension-leg lumen of the one-or-more extension-leg lumens extends through an entirety of the extension leg from a proximal end of the extension leg to a distal end of the extension leg.

Each extension-leg connector of the one-or-more extension-leg connectors 118 is over a proximal portion of an extension leg of the one-or-more extension legs 116. For example, each extension-leg connector of the one-or-more extension-leg connectors 118 can be a Luer connector over a proximal portion of an extension leg of the one-or-more extension legs 116. Through such an extension-leg connector, a corresponding extension leg and the extension-leg lumen thereof can be connected to another medical device and a lumen thereof. However, in the ready-to-operate state of the RICC insertion assembly 100 at least one extension-leg connector (e.g., the extension-leg connector including part of the primary lumen 128 of the RICC 102) is connected to the swivel-arm connector 194 of the swivel arm 182 of the coupler 108 to enforce the loop in the access guidewire 106 and the RICC 102 thereover.

As shown, the RICC 102 is a triluminal RICC including a set of three lumens; however, the RICC 102 is not limited to the set of the three lumens as set forth above. The set of three lumens includes a primary lumen 128, a secondary lumen 130, and a tertiary lumen 132 formed of fluidly connected portions of three catheter-tube lumens, three catheter-hub lumens, and three extension-leg lumens. The primary lumen 128 has a primary-lumen aperture 134 in the distal end of the first section 120 of the catheter tube 112, which corresponds to the distal end of the catheter tube 112 and a distal end of the RICC 102. The secondary lumen 130 has a secondary-lumen aperture 136 in a side of the distal portion of the catheter tube 112. The tertiary lumen 132 has a tertiary-lumen aperture 138 in the side of the distal portion of the catheter tube 112 proximal of the secondary-lumen aperture 136.

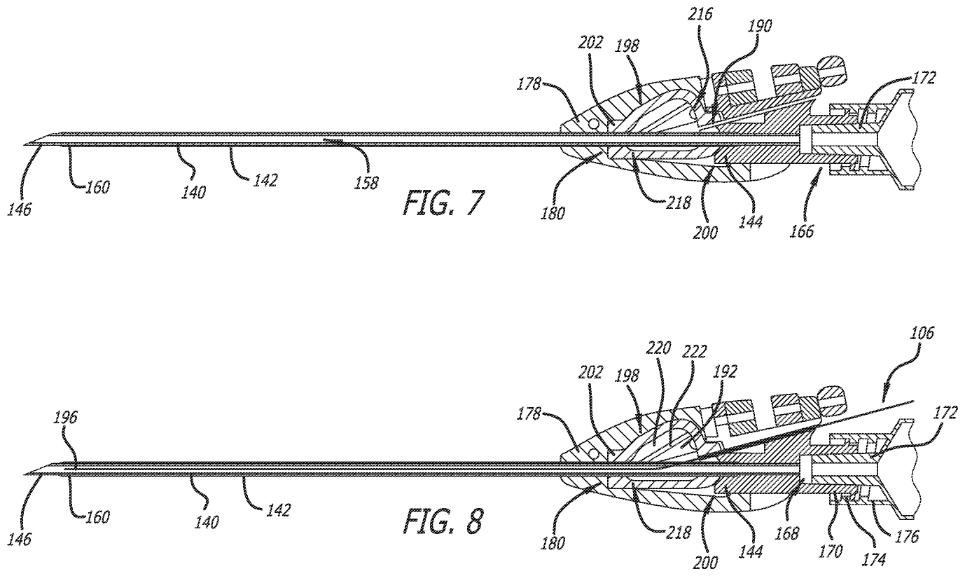

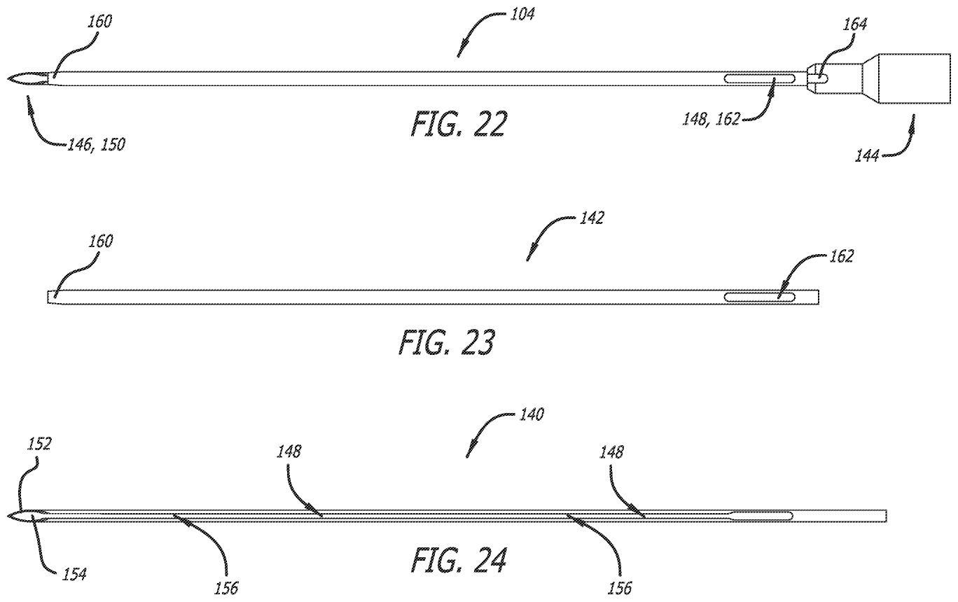

FIGS. 7, 8, and 22-24 illustrate various views of the introducer needle 104 of the RICC insertion assembly 100 in accordance with some embodiments.

As shown, the introducer needle 104 includes a needle shaft 140, a sheath 142 over the needle shaft 140, and a needle hub 144 in a proximal portion of the introducer needle 104 over both a proximal portion of the needle shaft 140 and a proximal portion of the sheath 142. In at least the ready-to-operate state of the RICC insertion assembly 100, the needle shaft 140 and the sheath 142 extend from the needle hub 144, through the sealing module 180, and out a distal end of the coupler housing 178.

The needle shaft 140 includes a needle tip 146 in a distal portion of the needle shaft 140 and a longitudinal needle slot 148 extending from the proximal portion of the needle shaft 140 through the needle tip 146.

The needle tip 146 includes a bevel 150 having a tip bevel 152 and a primary bevel 154 proximal of the tip bevel 152. A tip-bevel angle of the tip bevel 152 is greater than a primary-bevel angle of the primary bevel 154 such that the bevel 150 provides a smooth transition over the needle tip 146. Such a needle tip is thusly configured for establishing a needle tract from an area of skin into a blood-vessel lumen of a patient in accordance with the needle tract-establishing step of the method set forth below.

The needle slot 148 extends from the proximal portion of the needle shaft 140 through the needle tip 146, thereby forming a needle channel 156 along a majority of a length of the needle shaft 140 as opposed to a needle lumen therethrough. The needle slot 148 has a width sized in accordance with an outer diameter of the access guidewire 106, which allows the access guidewire 106 to pass from the proximal portion of the needle shaft 140 through the needle tip 146 when the introducer needle-withdrawing step of the method set forth below is performed.

While the needle shaft 140 includes the foregoing needle slot 148, it should be understood the introducer needle 104 includes a needle lumen 158; however, the needle lumen 158 results from the combination of the needle shaft 140 and the sheath 142 over the needle shaft 140. Indeed, the sheath 142 over the needle shaft 140 seals the needle slot 148 thereunder forming the needle lumen 158 of the introducer needle 104 and enabling the syringe 110 to aspirate blood in accordance with the blood-aspirating step of the method set forth below.

The sheath 142 includes a sheath tip 160 in a distal portion of the sheath 142 and a sheath opening 162 in a side of the proximal portion of the sheath 142.

The sheath tip 160 includes a relatively short taper from an outer diameter of the distal portion of the sheath 142 to an outer diameter of a distal end of the sheath 142, the latter of which is commensurate with an outer diameter of the distal portion of the needle shaft 140. The taper has a taper angle less than the primary-bevel angle of the primary bevel 154 of the needle tip 146, which, in turn, is less than the tip-bevel angle of the tip bevel 152 of the needle tip 146. The sheath tip 160 including such a taper is configured to provide a smooth transition from the needle tip 146 to the sheath body for the needle tract-establishing step of the method set forth below.

The sheath opening 162 opens to the needle slot 148 of the needle shaft 140 allowing the access guidewire 106 to pass through the sheath opening 162 and into the needle slot 148 in the ready-to-operate state of the RICC insertion assembly 100. Thus, the sheath opening 162 has a width approximately commensurate with a width of the needle slot 148, which, in turn, is sized in accordance with the diameter of the access guidewire 106. The sheath opening 162 also has a length sufficient to allow the access guidewire 106 to pass through the sheath opening 162 and into the needle slot 148 while also accommodating the blade 192 of the sealing module 180 under a distal end of the sheath opening 162. Notably, the sheath 142 over the needle shaft 140 seals the needle slot 148 thereunder except for that under the sheath opening 162. However, the sealing module 180 seals over the needle slot 148 exposed by the sheath opening 162 by sealing the proximal portions of the needle shaft 140 and the sheath 142 therein, thereby enabling the syringe 110 to aspirate blood in accordance with the blood-aspirating step of the method set forth below.

The sheath 142, or a sheath body thereof, is formed of a polymeric material configured to facilitate a smooth, consistent insertion of the introducer needle 104 from an area of skin to a blood-vessel lumen of a patient in accordance with the needle tract-establishing step of the method set forth below. In addition, the polymeric material has mechanical properties at a thickness of the sheath 142 sufficient to withstand collapse of the sheath 142 into the needle slot 148 of the needle shaft 140 when the blood-aspirating step of the method set forth below is performed, notably, while also facilitating the cutting of the sheath 142 off the needle shaft 140 in accordance with the introducer needle-withdrawing step of the method set forth below. Such a polymeric material can include, but is not limited to, polyethylene, polypropylene, polyurethane, or polytetrafluoroethylene.

The needle hub 144 includes an access-guidewire channel 164 in a distal portion of the needle hub 144 and a needle-hub connector 166 in a proximal portion of the needle hub 144.

The access-guidewire channel 164 of the needle hub 144 is configured to allow the access guidewire 106 to pass over the needle hub 144 and direct the access guidewire 106 into the access-guidewire passageway 190 of the sealing module 180. The access-guidewire channel 164 is open such that the access guidewire 106 lies in the access-guidewire channel 164 in at least the ready-to-operate state of the RICC insertion assembly 100. Advantageously, the open access-guidewire channel 164 allows the access guidewire 106 to remain in place when the introducer needle 104 is withdrawn from the RICC insertion assembly 100 in accordance with the introducer needle-withdrawing step of the method set forth below.

Notably, the distal portion of the needle hub 144 including the access-guidewire channel 164 can be configured as the follower of the sealing module 180 set forth below. The needle hub 144 including the follower is configured to be removably disposed in the needle-hub receptacle 200 of the coupler housing 178. As set forth below, the follower axially compresses the sealing-module insert 202 in the sealing-module cavity 198 when the needle hub 144 is disposed in the needle-hub receptacle 200. Axial compression of the sealing-module insert 202 in the sealing-module cavity 198, in turn, radially compresses the sealing-module insert 202 in the sealing-module cavity 198.

The needle-hub connector 166 includes a needle-hub bore 168 and an optional needle-hub flange 170 about the needle-hub connector 166.

The needle-hub bore 168 of the needle-hub connector 166 is configured to accept a syringe tip 172 of the syringe 110 therein for fluidly connecting the introducer needle 104 to the syringe 110. Indeed, the needle-hub bore 168 can have a Luer taper (e.g., a 6% taper) configured to accept the syringe tip 172 therein, which syringe tip 172 can be complementarily configured with a Luer taper.

The needle-hub flange 170 of the needle-hub connector 166 is configured to screw together with internal threads 174 of a threaded collar 176 around the syringe tip 172 of the syringe 110. While the threaded collar 176 of the syringe 110 is optional, the needle-hub flange 170 advantageously provides a so-called Luer lock-style connection with the internal threads 174 of the threaded collar 176 when both are present. This provides added security against inadvertent disconnection of the introducer needle 104 and the syringe 110 over that provided by an otherwise Luer slip-style connection.

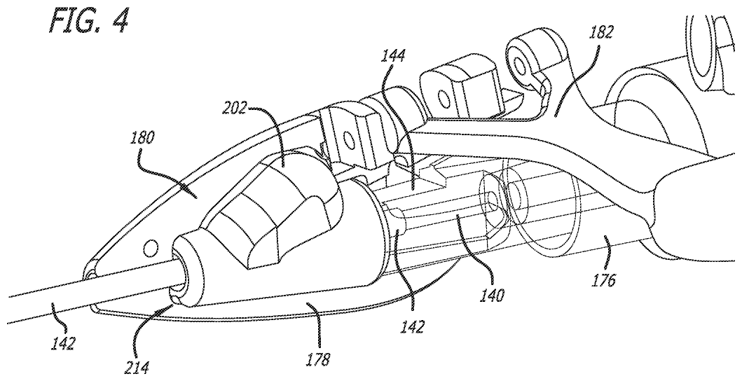

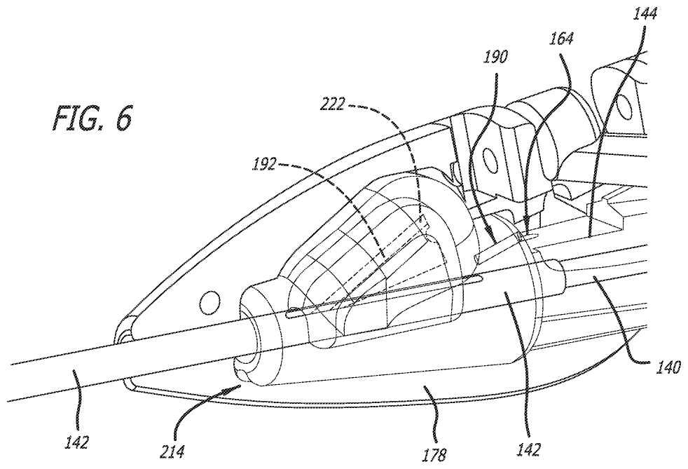

FIGS. 4-8 illustrate various view of the coupler 108 of the RICC insertion assembly 100 in accordance with some embodiments.

As shown, the coupler 108 includes a coupler housing 178, a sealing module 180, and a swivel arm 182 swivelably coupled to the coupler housing 178.

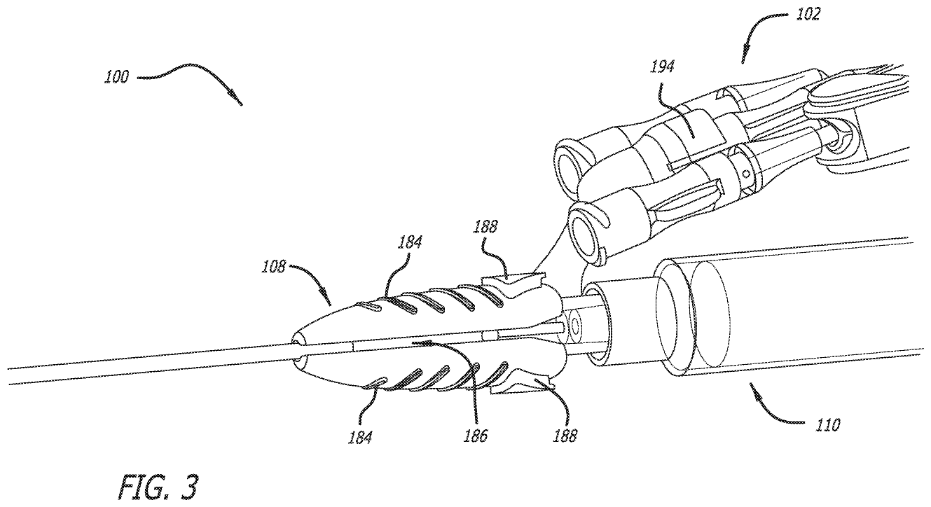

The coupler housing 178 includes two molded halves coupled together to form an ovoid body configured to be comfortably held underhand (e.g., cradled) or overhand in either a left hand for a left-handed venipuncture or a right hand for a right-handed venipuncture with the RICC insertion assembly 100. To further facilitate such venipunctures, an outside of each half of the two molded halves can be textured as shown with grip-enhancing arcuate ridges 184 or the like. An inside of each half of the two molded halves includes depressions that form a sealing-module cavity 198 and a needle-hub receptacle 200 proximal of the sealing-module cavity 198 when the two molded halves are coupled together as shown. (See FIGS. 4-8, which include the sealing-module insert 202 disposed in a depression of a molded half of the two molded halves that form the sealing-module cavity 198. FIGS. 4-8 also include the needle hub 144 of the introducer needle 104 disposed in another depression of the molded half of the two molded halves that form the needle-hub receptacle 200.) In addition, each half of the two molded halves includes a lock-button through hole for a corresponding lock button of the pair of lock buttons 188 of the needle-hub lock. (See FIG. 3 for the pair of lock buttons 188 extending through the corresponding pair of lock-button through holes.) Notably, the coupler housing 178 includes a longitudinal coupler-housing slot 186 formed between the two molded halves. The coupler-housing slot 186 is configured to allow the access guidewire 106 to escape from the coupler housing 178 when the introducer needle 104 is withdrawn from the coupler 108 in the introducer needle-withdrawing step of the method set forth below. Notwithstanding the foregoing, it should be understood the coupler housing 178 can be made from one piece, two different pieces than the foregoing molded halves, or a more than two pieces. In addition, the lock buttons 188 can be configured with different shapes and locations than that shown in FIG. 3, optionally with a different configuration for locking and unlocking the needle-hub lock. The needle-hub lock can also be implemented through other means such as levers, different locks, or switches.

The sealing-module cavity 198 is configured to hold the sealing-module insert 202 therein. Indeed, the sealing-module cavity 198 includes the sealing-module insert 202 disposed therein in one or more states of the RICC insertion assembly 100 such as the ready-to-operate state or one or more operating states of the RICC insertion assembly 100. Notably, the sealing-module cavity 198 is further configured with sufficient space to allow the sealing-module insert 202 set forth below to relax and separate for the escape of the access guidewire 106 when the introducer needle 104 is withdrawn from the coupler 108 in the introducer needle-withdrawing step of the method set forth below.

The needle-hub receptacle 200 is configured to hold the needle hub 144 of the introducer needle 104 therein. Indeed, the needle-hub receptacle 200 includes the needle hub 144 inserted therein in the ready-to-operate state of the RICC insertion assembly 100. Notably, a needle-hub lock configured to lock the needle hub 144 in the needle-hub receptacle 200 is positioned about the needle-hub receptacle 200. A pair of lock buttons 188 (e.g., spring-loaded lock buttons) of the needle-hub lock is distributed between opposite sides of the coupler 108, particularly in the lock-button through holes of the two molded halves of the coupler housing 178 such that each lock button of the pair lock buttons 188 extends through the coupler housing 178 on its respective side of the coupler 108. The lock buttons 188 are configured to unlock the needle hub 144 when the lock buttons 188 are pressed into the coupler 108 for withdrawal of the introducer needle 104 from the coupler 108. When the distal portion of the needle hub 144 is configured as the follower of the sealing module 180, unlocking the lock buttons 188 can immediately release the axial compression compressing the sealing-module insert 202 in the sealing-module cavity 198. This allows the sealing-module insert 202 to relax for withdrawing the introducer needle 104 from the coupler 108. The also allows the sealing-module to separate for the escape of the access guidewire 106 when the introducer needle 104 is withdrawn from the coupler 108 in the introducer needle-withdrawing step of the method set forth below.

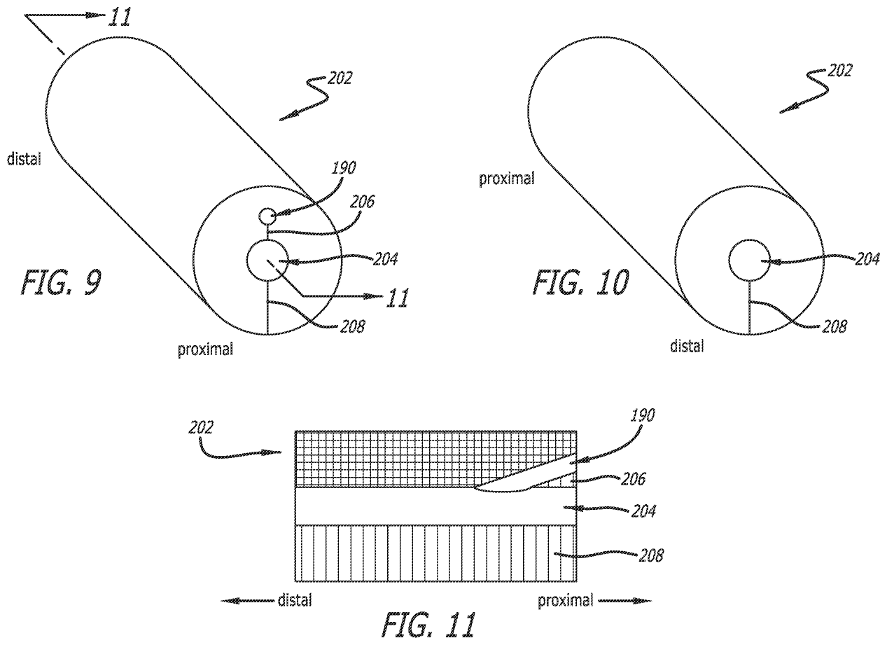

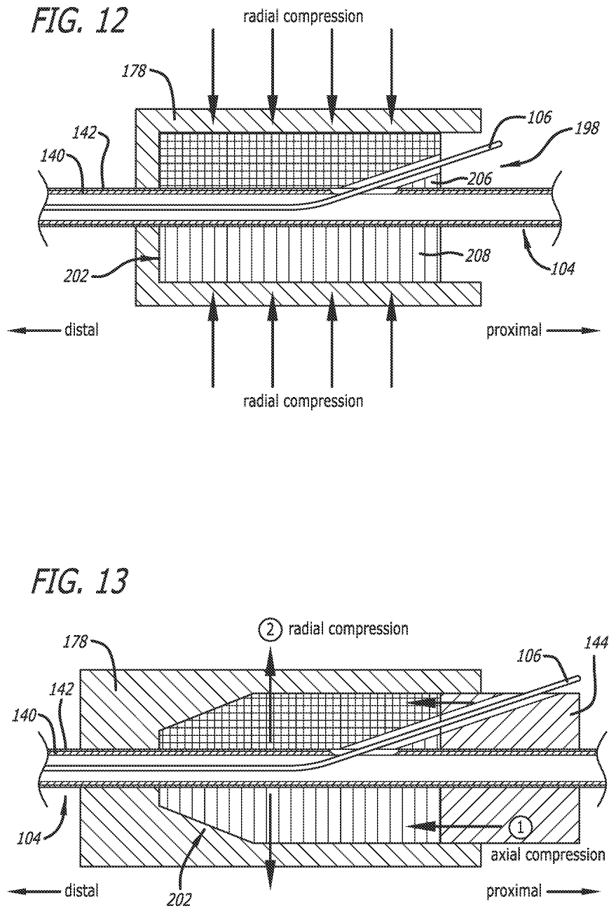

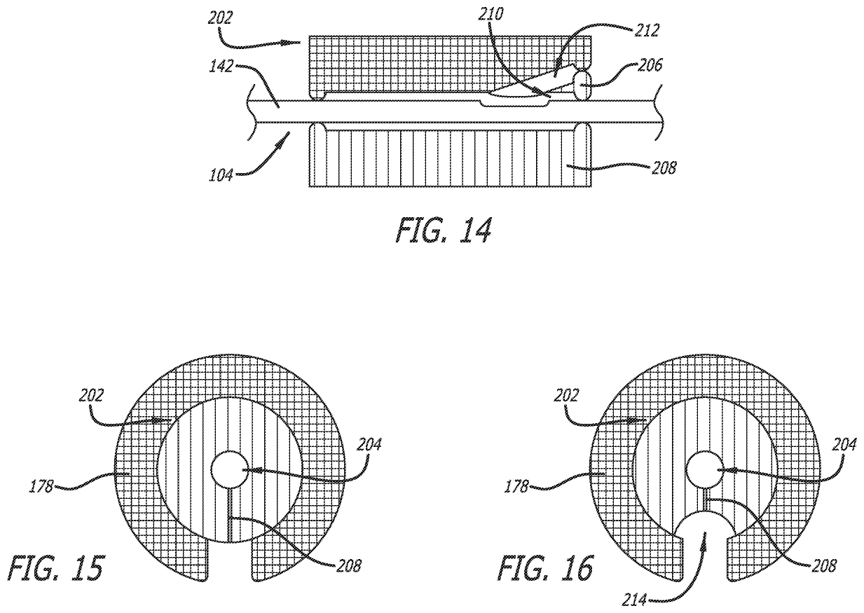

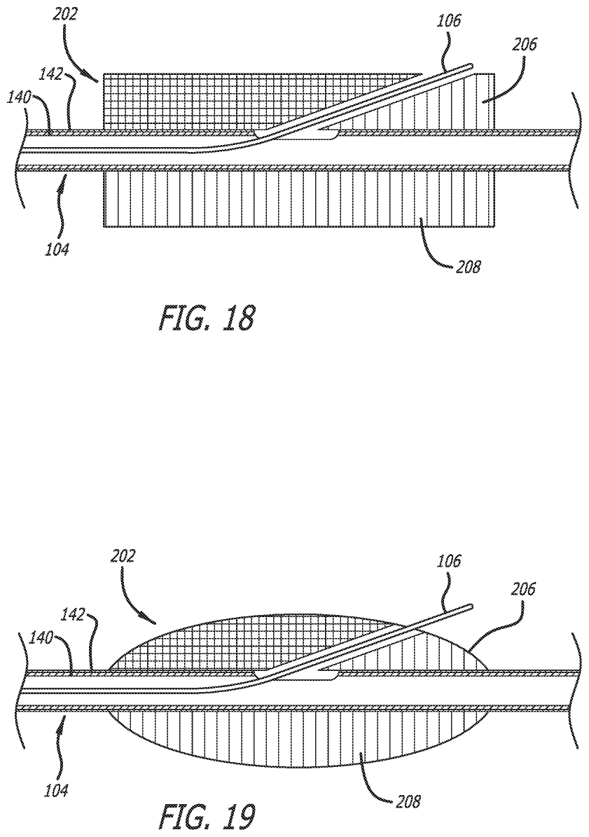

FIGS. 9-11, 14, and 18-20 illustrate various views of an elastomeric (e.g., silicone) sealing-module insert 202 of the sealing module 180 in accordance with some embodiments. FIGS. 12, 13, and 15-17 illustrate the sealing-module in the sealing-module cavity 198 of the coupler housing 178 in accordance with some embodiments.

As shown, the sealing-module insert 202 is disposed in or otherwise inserted into the sealing-module cavity 198 of the coupler housing 178 where the sealing-module insert 202 can be compressed to seal around the introducer needle 104 and the access guidewire 106. Indeed, the sealing-module insert 202 can be radially compressed in the sealing-module cavity 198 or both axially and radially compressed in the sealing-module cavity 198 to seal around the introducer needle 104 and the access guidewire 106. With seals around the introducer needle 104 and the access guidewire 106 in accordance with the foregoing, the syringe 110 is able to aspirate blood in accordance with the blood-aspirating step of the method set forth below.

In an example of radial compression, the sealing-module insert 202 can be radially compressed in the sealing-module cavity 198 by the coupler housing 178 itself as shown in FIG. 12. Without limiting such embodiments, the two halves of the coupler housing 178 can be coupled together like a clamshell by way of a hinge opposite the coupler-housing slot 186 formed between the two halves of the coupler housing 178. One or more clamps across the coupler-housing slot 186 can be clamped in at least the ready-to-operate state of the RICC insertion assembly 100 to hold the two halves of the coupler housing 178 together, apply sufficient pressure to radially compress the sealing-module insert 202 in the sealing-module cavity 198, and seal the sealing-module insert 202 around the introducer needle 104 and the access guidewire 106. The one-or-more clamps can be unclamped in the one-or-more operating states of the RICC insertion assembly 100 to relieve the radial compression and allow the sealing-module insert 202 to relax for the escape of the access guidewire 106 through the slits 206 and 208 set forth below when the introducer needle 104 is withdrawn from the coupler 108.

In an example of both radial and axial compression, the sealing-module insert 202 can be both axially and radially compressed in the sealing-module cavity 198 by a follower of the sealing module 180 proximal of the sealing-module insert 202 as shown in FIGS. 13 and 21. Without limiting such embodiments, the follower can be the distal portion of the needle hub 144, which axially compresses the sealing-module insert 202 in the sealing-module cavity 198 when the needle hub 144 is disposed in the needle-hub receptacle 200 such as in the ready-to-operate state of the RICC insertion assembly 100. Axial compression of the sealing-module insert 202 in the sealing-module cavity 198, in turn, radially compresses the sealing-module insert 202 in the sealing-module cavity 198, thereby sealing the sealing-module insert 202 around the introducer needle 104 and the access guidewire 106. The needle hub 144 and, thus, the follower of the sealing module 180, can be removed from the needle-hub receptable in the one-or-more operating states of the RICC insertion assembly 100 to relieve both the axial and the radial compression and allow the sealing-module insert 202 to relax for the escape of the access guidewire 106 through the slits 206 and 208 set forth below when the introducer needle 104 is withdrawn from the coupler 108.

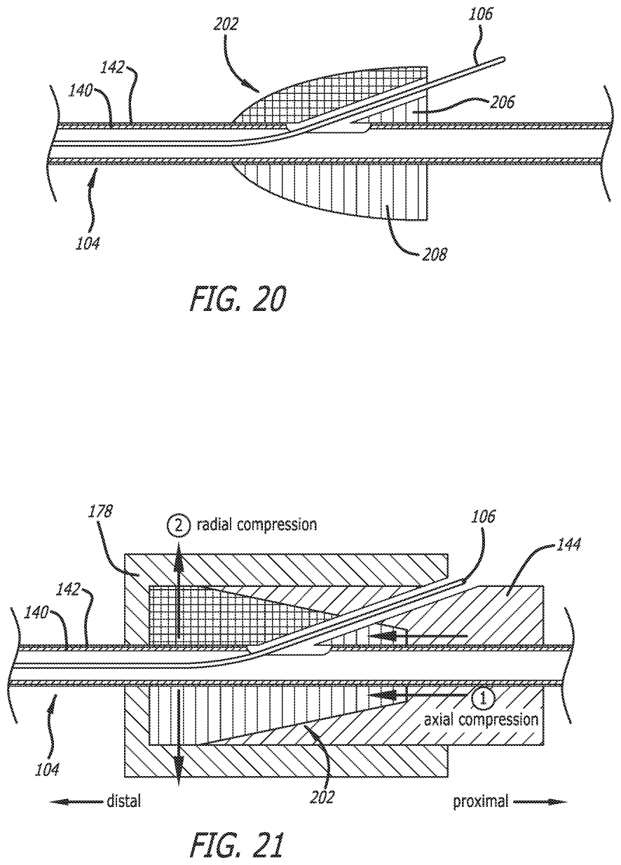

The sealing-module insert 202 and the sealing-module cavity 198 in which the sealing-module insert 202 is disposed can have any of a number of corresponding shapes. In an example, the sealing-module insert 202 can be shaped like a cylinder and the sealing-module cavity 198 correspondingly shaped for disposal of the sealing-module insert 202 therein. In addition to FIGS. 9, 10, and 18, such a sealing-module insert is shown in FIG. 12 without the needle hub 144 or the follower thereof as the sealing-module insert 202 can be radially compressed in the sealing-module cavity 198 by the coupler housing 178 itself in such embodiments. In another example, the sealing-module insert 202 can be shaped like a tapered cylinder and the sealing-module cavity 198 correspondingly shaped for disposal of the sealing-module insert 202 therein. Such a sealing-module insert is shown in FIG. 13 as well as FIG. 21; however, a tapered portion of the sealing-module insert 202 of FIG. 13 tapers in a distal direction, whereas the tapered portion of the sealing-module insert 202 of FIG. 21 tapers in a proximal direction. The needle hub 144 or the follower thereof, which can axially compress the sealing-module insert 202 in the sealing-module cavity 198 in such embodiments, can be correspondingly shaped for axially compressing the sealing-module insert 202 in the sealing-module cavity 198. Further examples of at least the sealing-module insert 202 are shown in FIGS. 19 and 20, wherein the sealing-module insert 202 is ovoid like the coupler housing 178 or a frustrum of the ovoid sealing-module insert 202. It should be appreciated that the passageways 190 and 204 and the slits 206 and 208 set forth below can vary in accordance with the shape of at least the sealing-module insert 202.

The sealing-module insert 202 includes a pair of passageways 190 and 204, thereby configuring the sealing-module insert 202 to separately seal around the proximal portion of the introducer needle 104 and the distal portion of the access guidewire 106 when the sealing-module insert 202 is compressed in the sealing-module cavity 198 in one or more states of the RICC insertion assembly 100 (e.g., the ready-to-operate state or the one-or-more operating states of the RICC insertion assembly 100). Indeed, the passageways 190 and 204 of the sealing-module insert 202 include an introducer-needle passageway 204 and an access-guidewire passageway 190. The introducer-needle passageway 204 is configured to seal around the proximal portion of the introducer needle 104 when the sealing-module insert 202 is compressed in the sealing-module cavity 198, and the access-guidewire passageway 190 is configured to seal around the distal portion of the access guidewire 106 when the sealing-module insert 202 is compressed in the sealing-module cavity 198. With seals around the introducer needle 104 and the access guidewire 106 in accordance with the foregoing, the syringe 110 is able to aspirate blood in accordance with the blood-aspirating step of the method set forth below.

As opposed to the introducer-needle passageway 204, which passes through both proximal and distal ends of the sealing-module insert 202, the access-guidewire passageway 190 passes through the proximal end of the sealing-module insert 202. A distal end of the access-guidewire passageway 190 connects to a medial portion of the introducer-needle passageway 204, thereby allowing the distal portion of the access guidewire 106 to be disposed in the needle shaft 140 through the needle slot 148. Indeed, the access-guidewire passageway 190 is configured to direct the access guidewire 106 from the access-guidewire channel 164 of the needle hub 144 into both the sheath opening 162 of the sheath 142 and the needle slot 148 of the needle shaft 140 thereunder such that the access guidewire 106 can be disposed in the introducer needle 104 with the distal end of the access guidewire 106 just proximal of the needle tip 146 in the ready-to-operate state of the RICC insertion assembly 100.

The sealing-module insert 202 includes a pair of slits 206 and 208, thereby configuring the sealing-module insert 202 to at least partially separate when allowed to relax in the sealing-module cavity 198 of the coupler housing 178. The slits 206 and 208 allow the access guidewire 106 to escape from both the access-guidewire passageway 190 and the introducer-needle passageway 204 after the introducer needle 104 is withdrawn from at least the introducer-needle passageway 204 of the sealing-module insert 202 when performing the introducer needle-withdrawing step of the method set forth below to remove the introducer needle 104 from the RICC insertion assembly 100. Indeed, the slits 206 and 208 in the sealing-module insert 202 include a partial-length longitudinal slit 206 and a full-length longitudinal slit 208, wherein the partial-length longitudinal slit 206 has a slit length less than a full length of the sealing-module insert 202 and the full-length longitudinal slit 208 has a slit length equal to the length of the sealing-module insert 202. The partial-length longitudinal slit 206 is between the access-guidewire passageway 190 and the introducer-needle passageway 204, which allows the access guidewire 106 to escape from the access-guidewire passageway 190 into the introducer-needle passageway 204 after the introducer needle 104 is withdrawn from the RICC insertion assembly 100 through the introducer-needle passageway 204. The full-length longitudinal slit 208 is between the introducer-needle passageway 204 and an exterior of the sealing-module insert 202, which subsequently allows the access guidewire 106 to escape from the introducer-needle passageway 204 after the introducer needle 104 is withdrawn from the RICC insertion assembly 100 through the introducer-needle passageway 204. Notably, the full-length longitudinal slit 208 opens into the coupler-housing slot 186 of the coupler housing 178, which, as set forth above, is configured to allow the access guidewire 106 to escape from the coupler housing 178 after the introducer needle 104 is withdrawn from the RICC insertion assembly 100 through the introducer-needle passageway 204.

FIG. 14 illustrates a longitudinal cross section of the sealing-module insert 202 with internal reliefs 210 and 212 in the introducer-needle passageway 204 and the access-guidewire passageway 190 of the sealing-module insert 202 in accordance with some embodiments.

As shown, the introducer-needle passageway 204 of the sealing-module insert 202 can include an internal relief 210 between proximal and distal portions of the introducer-needle passageway 204 to reduce friction on the introducer needle 104 when being withdrawn from the coupler 108 and the sealing module 180 thereof in the introducer needle-withdrawing step of the method set forth below. Additionally or alternatively, the access-guidewire passageway 190 can include an internal relief 212 in at least a proximal portion of the access-guidewire passageway 190 to reduce friction on the access guidewire 106 when being advanced into the coupler 108 and the sealing module 180 thereof in the access guidewire-advancing step of the method set forth below. With the internal relief 210 between proximal and distal portions of the introducer-needle passageway 204, only proximal- and distal-end portions of the introducer-needle passageway 204 including proximal and distal ends of the introducer-needle passageway 204 are configured to seal around the proximal portion of the introducer needle 104 when the sealing-module insert 202 is compressed in the sealing-module cavity 198. With the internal relief 212 in the proximal portion of the access-guidewire passageway 190, only a proximal-end portion of the access-guidewire passageway 190 including a proximal end of the access-guidewire passageway 190 is configured to seal around the distal portion of the access guidewire 106 when the sealing-module insert 202 is compressed in the sealing-module cavity 198. Notably, while the internal reliefs 210 and 212 are configured to respectively reduce friction of the introducer-needle passageway 204 and the access-guidewire passageway 190 on the introducer needle 104 and the access guidewire 106, respectively, a lubricant can be additionally or alternatively employed in the sealing-module insert 202 to reduce the friction.

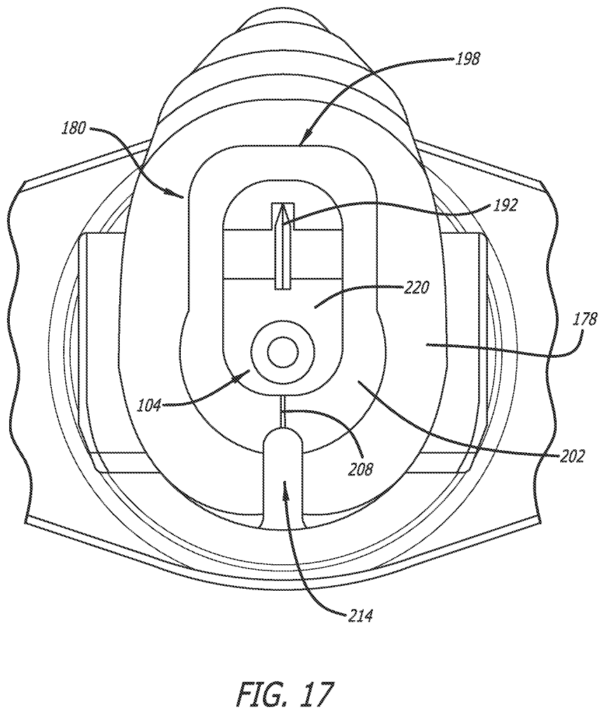

FIGS. 16 and 17 illustrates a transverse cross section of at least the distal portion of the sealing module 180 with an external relief between the sealing-module insert 202 and the coupler housing 178 in accordance with some embodiments.

As shown, the sealing-module insert 202 can include an external relief 214 between the sealing-module insert 202 and the coupler housing 178, wherein the external relief 214 has a length less than the full-length longitudinal slit 208 up to coextensive with the full-length longitudinal slit 208 in the sealing-module insert 202. The external relief 214 is configured to allow the access guidewire 106 to escape from the introducer-needle passageway 204 without binding the access guidewire 106 in the full-length longitudinal slit 208.

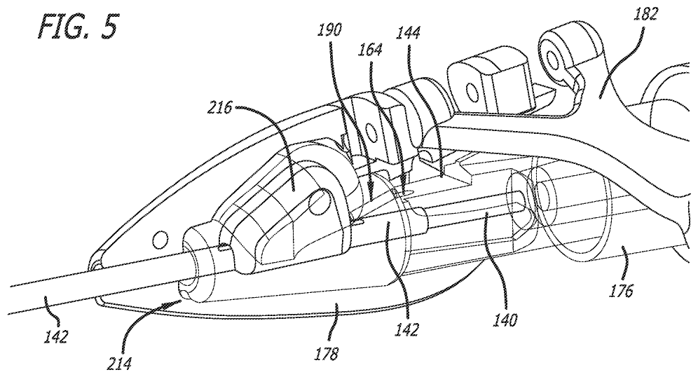

FIG. 17 also illustrates a transverse cross section of a blade module 216 of the sealing module 180 in accordance with some embodiments. FIGS. 5-8 illustrate other views of the blade module 216 in accordance with some embodiments.

As shown, the blade module 216 is disposed in a sealing module-insert cavity 218 of the sealing-module insert 202 with a blade 192 of the blade module 216 extending from a blade holder 220 of the blade module 216. Notwithstanding the foregoing, however, the blade 192 can alternatively be overmolded into the sealing-module insert 202, thereby integrating the blade 192 in the sealing-module insert 202 instead of disposing the blade module 216 in the sealing module-insert cavity 218. Regardless, the blade 192 or a blade tip thereof is disposed in the needle slot 148 under a distal end of the sheath opening 162 in at least a ready-to-operate state of the RICC insertion assembly 100. The blade 192 includes a distal-facing blade edge 222 configured to cut the sheath 142 away from the needle shaft 140 as the introducer needle 104 is withdrawn from the coupler 108 through the introducer-needle passageway 204 in the introducer needle-withdrawing step of the method set forth below. Cutting the sheath 142 away from the needle shaft 140 as the introducer needle 104 is withdrawn from the coupler 108 allows the access guidewire 106 to escape from the needle shaft 140 by way of the needle slot 148.

The swivel arm 182 includes a swivel-arm connector 194 connected to an extension-leg connector of the one-or-more extension-leg connectors 118 in the ready-to-operate state of the RICC insertion assembly 100. While not shown, the swivel-arm connector 194 includes an access-guidewire attachment point within the swivel-arm connector 194 to which the proximal end of the access guidewire 106 is attached in the ready-to-operate state of the RICC insertion assembly 100. In combination with the distal end of the access guidewire 106 being disposed in the needle lumen 158 of the introducer needle 104, the loop in the access guidewire 106 set forth above is enforced. Advantageously, the swivel arm 182 is configured to flip the loop—or at least the one-or-more extension legs 116 of the RICC 102 thereof—between a sinistral side of the RICC insertion assembly 100 and a dextral side of the RICC insertion assembly 100 to accommodate both left-handed and right-handed venipunctures with the RICC insertion assembly 100. Indeed, the swivel arm 182 is configured to flip the loop from the sinistral side of the RICC insertion assembly 100 as shown in FIG. 1 to the dextral side of the RICC insertion assembly 100 to accommodate a left-handed venipuncture with the RICC insertion assembly 100. Likewise, the swivel arm 182 is configured to flip the loop from the dextral side of the RICC insertion assembly 100 to the sinistral side of the RICC insertion assembly 100 to accommodate a right-handed venipuncture with the RICC insertion assembly 100. Notwithstanding the foregoing, it should be understood the swivel arm 182 is but one example of an extension arm for connecting the foregoing extension-leg connector of the one-or-more extension-leg connectors 118 and the proximal end of the access guidewire 106. Indeed, other configurations of the extension arm besides the swivel arm 182 are contemplated including a fixed-position arm, wherein the extension-leg connector of the one-or-more extension-leg connectors 118 and the proximal end of the access guidewire 106 can be in a fixed positioned on a patient-facing side of the RICC insertion assembly 100, opposite the patient-facing side of the RICC insertion assembly 100 on a clinician-facing side of the RICC insertion assembly 100, the sinistral side of the RICC insertion assembly 100, or the dextral side of the RICC insertion assembly 100.

FIGS. 1, 2, and 8 illustrate various view of the access guidewire 106 of the RICC insertion assembly 100 in accordance with some embodiments.

The access guidewire 106 includes a proximal portion including a proximal end and a distal portion including a distal end. In the ready-to-operate state of the RICC insertion assembly 100, the proximal end of the access guidewire 106 is coupled to the swivel arm 182, particularly the access-guidewire attachment point within the swivel-arm connector 194 of the swivel arm 182. In addition, the proximal portion of the access guidewire 106 extends along the primary lumen 128 of the RICC 102. The distal portion of the access guidewire 106 also extends along the primary lumen 128 of the RICC 102, but the distal portion of the access guidewire 106 further extends out the distal end of the RICC 102, into the sealing module 180 over the needle hub 144 by way of the access-guidewire channel 164, into the needle shaft 140 through both the sheath opening 162 of the sheath 142 and the needle slot 148 of the needle shaft 140, and along the needle lumen 158 of the introducer needle 104 in the ready-to-operate state of the RICC insertion assembly 100. As shown in FIG. 8, the distal end of the access guidewire 106 is disposed in the needle lumen 158 just proximal of the needle tip 146 in the ready-to-operate state of the RICC insertion assembly 100. Again, the proximal and distal ends of the access guidewire 106 enforce the loop in the access guidewire 106 in the ready-to-operate state of the RICC insertion assembly 100, which loop the RICC 102 is disposed over, thereby keeping the RICC insertion assembly 100 in a relatively compact form.

The access guidewire 106 can include a guidewire tip 196 in the distal portion of the access guidewire 106, which adopts a T shape configured to prevent puncturing a back wall of a blood vessel. Such a guidewire tip assumes a straightened state in the ready-to-operate state of the RICC insertion assembly 100 and a curved state when the guidewire tip 196 is advanced beyond the needle tip 146 (e.g., advanced into a blood-vessel lumen) in a deployed state of the RICC insertion assembly 100.

The access guidewire 106 can further include a bare-wire portion and a wound-wire portion distal of the bare-wire portion, proximal of the bare-wire portion, or both. While not shown, the bare-wire portion, when present, distally extends through the access-guidewire passageway 190 of the sealing module 180 in at least the ready-to-operate state of the RICC insertion assembly 100 such that the sealing module 180 forms a fluid-tight seal around the bare-wire portion of the access guidewire 106. Notably, the foregoing bare-wire portion can instead be a flat-wound or ground-wound portion of the access guidewire 106, wherein the flat-wound portion includes windings of a tape instead of a round wire, and wherein the ground-wound portion includes windings of a round wire ground down to flatten the windings.

### Methods

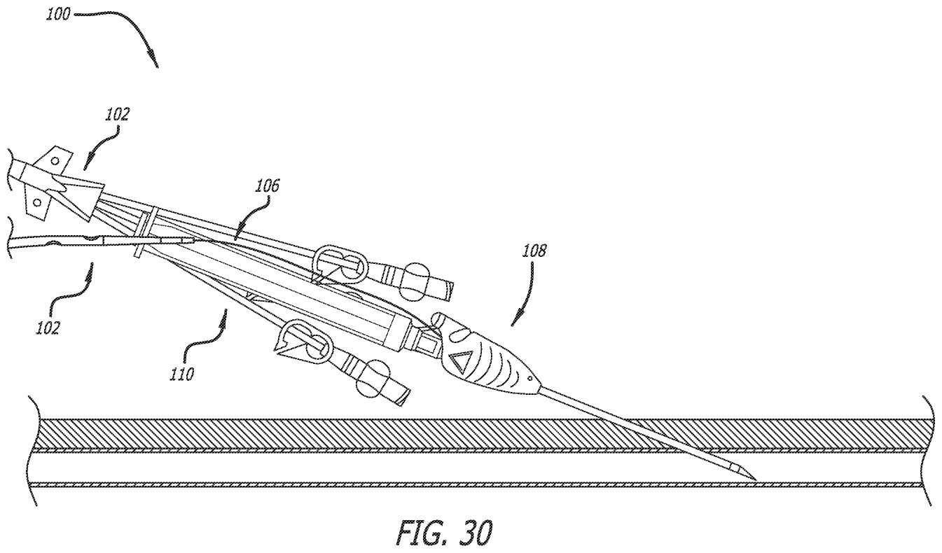

FIGS. 30-34 illustrate various steps a method of using the RICC insertion assembly 100 in accordance with some embodiments.

As shown, methods of the RICC insertion assembly 100 include a method for inserting the RICC 102 into a blood-vessel lumen of a patient. Such a method includes one or more steps selected from an insertion assembly-obtaining step, a needle tract-establishing step, a blood-aspirating step, an access guidewire-advancing step, an unlocking step, an introducer needle-withdrawing step, a RICC-advancing step, an access guidewire-withdrawing step, a maneuver guidewire-advancing step, another RICC-advancing step, and a maneuver guidewire-withdrawing step.

The insertion assembly-obtaining step includes obtaining the RICC insertion assembly 100, optionally, already in the ready-to-operate state thereof. If the RICC insertion assembly 100 is not in the ready-to-operate state upon obtaining it in the insertion assembly-obtaining step, the RICC insertion assembly 100 can be adjusted to put it in the ready-to-operate state for subsequent steps.

As set forth above, the RICC insertion assembly 100 includes the RICC 102, the introducer needle 104, the access guidewire 106, and the coupler 108 coupling the RICC 102 and the introducer needle 104 together in at least the ready-to-operate state of the RICC insertion assembly 100. The sealing module 180 of the coupler 108, which, again, is formed between at least the sealing-module cavity 198 of the coupler housing 178 and the sealing-module insert 202 disposed in the sealing-module cavity 198, separately seals around the proximal portion of the introducer needle 104 and the distal portion of the access guidewire 106 in at least the ready-to-operate state of the RICC insertion assembly 100. Within the sealing module 180, the distal portion of the access guidewire 106 is disposed in the needle shaft 140 through the needle slot 148. This is accomplished through a connection between the distal end of the access-guidewire passageway 190 and the medial portion of the introducer-needle passageway 204 in the sealing-module insert 202.

FIG. 30 illustrates the needle tract-establishing step of the method in accordance with some embodiments.

The needle tract-establishing step includes establishing a needle tract from an area of skin to the blood-vessel lumen with the introducer needle 104. The needle tract-establishing step can also include ensuring blood flashes back into the introducer needle 104 (e.g., the needle hub 144 of the introducer needle 104), the syringe 110 (e.g., the syringe tip 172, a barrel of the syringe 110, or both) fluidly connected to the introducer needle 104, or both the introducer needle 104 and the syringe 110, thereby confirming the needle tract extends into the blood-vessel lumen. To enhance blood flashback, a slight vacuum can be drawn with the syringe 110 while establishing the needle tract.

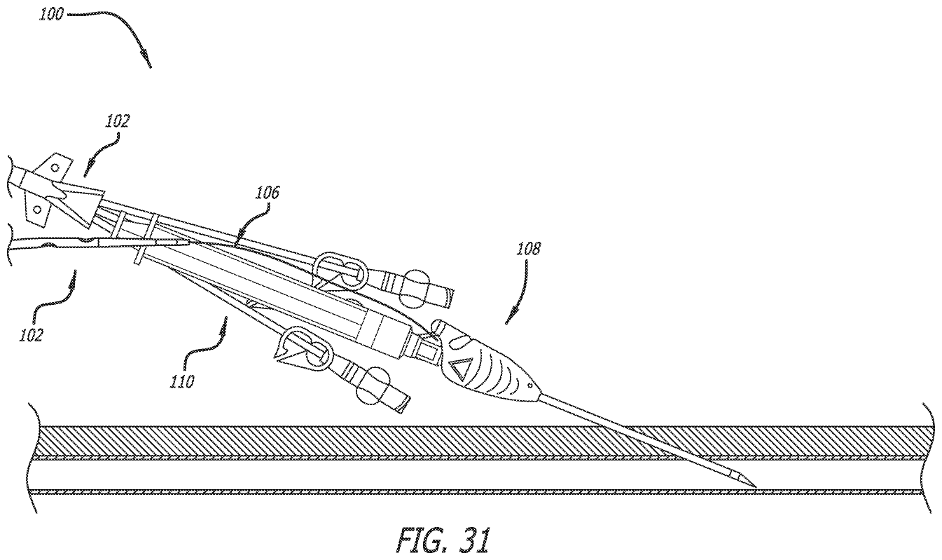

FIG. 31 illustrates the blood-aspirating step of the method in accordance with some embodiments.

The blood-aspirating step includes aspirating blood with the syringe 110 to confirm the needle tract extends into the blood-vessel lumen before withdrawing the introducer needle 104 from the coupler 108 in the introducer needle-withdrawing step. Again, the sheath 142 over the needle shaft 140 seals the needle slot 148 of the needle shaft 140 thereunder. In particular, the sheath 142 seals the needle slot 148 outside of the sealing module 180. The sealing module 180, in turn, seals over the sheath opening 162 of the sheath 142, which sheath opening 162 allows the access guidewire 106 to pass from the access-guidewire passageway 190 of the sealing-module insert 202 and into the needle shaft 140 by way of the needle slot 148. The sealing module 180 also seals around the distal portion of the access guidewire 106. Such seals enable the syringe 110 to aspirate blood in the blood-aspirating step.

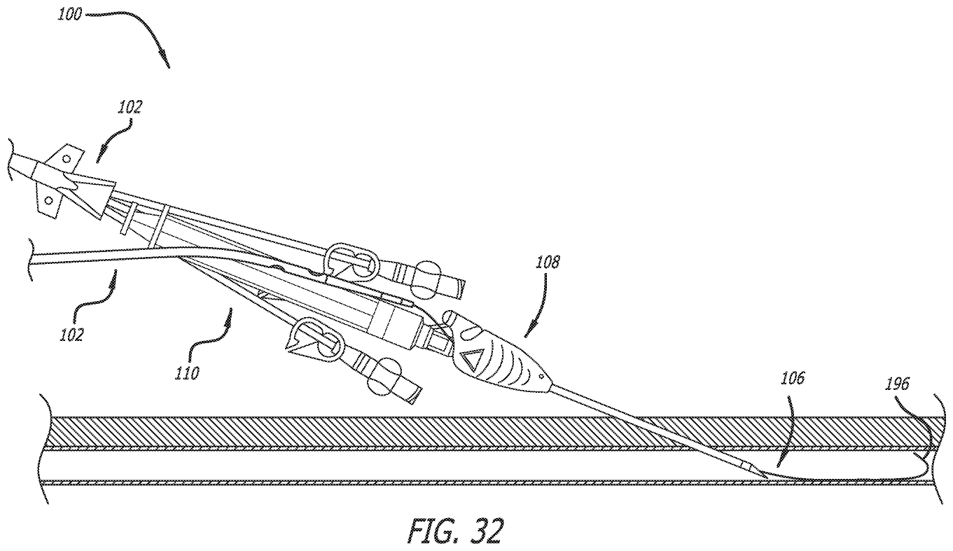

FIG. 32 illustrates the access guidewire-advancing step of the method in accordance with some embodiments.

The access guidewire-advancing step includes advancing the distal portion of the access guidewire 106 through both the sealing module 180 and the needle slot 148 of the needle shaft 140. In the access guidewire-advancing step, the distal end of the access guidewire 106 is advanced from its initial location in the introducer needle 104 just proximal of the needle tip 146 of the needle shaft 140 into the blood-vessel lumen.

The unlocking step includes pressing the pair of lock buttons 188 of the needle-hub lock, which lock buttons 188 are distributed between opposite sides of the coupler 108, into the coupler 108 to unlock the needle hub 144. The unlocking step is performed before the introducer needle-withdrawing step of withdrawing the introducer needle 104 from the coupler 108.

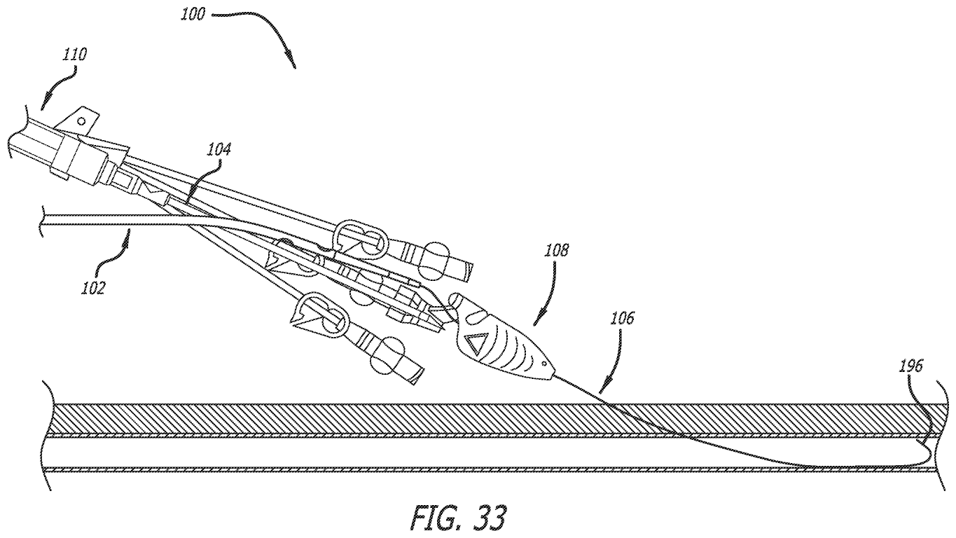

FIG. 33 illustrates the introducer needle-withdrawing step of the method in accordance with some embodiments.

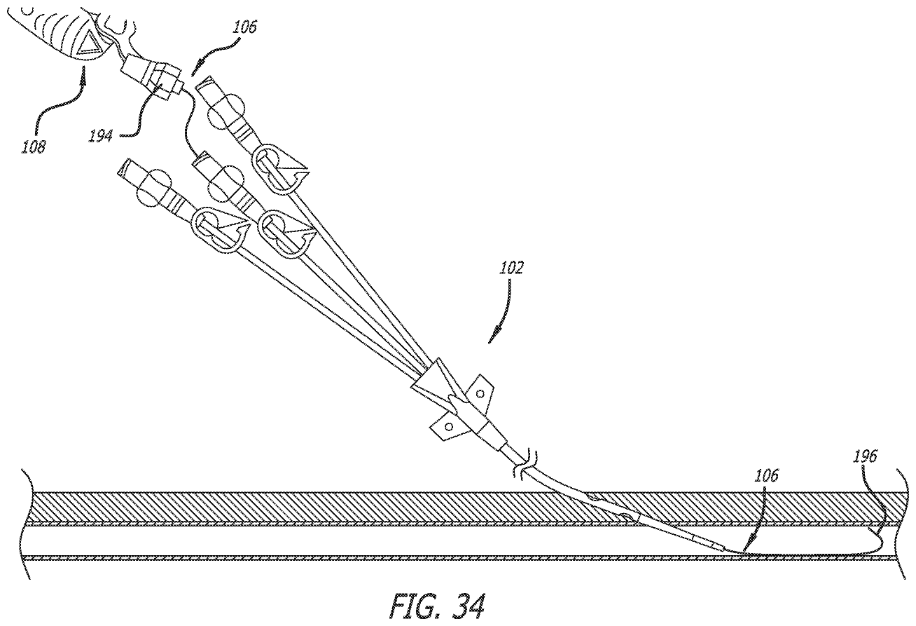

The introducer needle-withdrawing step includes withdrawing the introducer needle 104 from the coupler 108 leaving the access guidewire 106 in place in the blood-vessel lumen. Withdrawing the introducer needle 104 from the coupler 108 removes the needle hub 144 of the introducer needle 104 from the needle-hub receptacle 200 of the coupler housing 178, thereby removing both the axial and radial compression on the sealing-module insert 202 and unsealing the proximal portion of the introducer needle 104 and the distal portion of the access guidewire 106. Withdrawing the introducer needle 104 from the coupler 108 also includes cutting the needle slot-sealing sheath 142 of the introducer needle 104 away from the needle shaft 140. As set forth above, the sealing module 180 includes the blade 192 disposed in the needle slot 148 under the distal end of the sheath opening 162 of the sheath 142 with the distal-facing blade edge 222 for the cutting of the sheath 142 away from the needle shaft 140. The cutting of the sheath 142 away from the needle shaft 140 allows the access guidewire 106 to escape from the needle shaft 140 by way of the needle slot 148. In addition, the coupler-housing slot 186 of the coupler housing 178 allows the access guidewire 106 to escape from the coupler housing 178 after the withdrawing of the introducer needle 104 from the coupler 108. (See, for example, FIG. 34, wherein the distal portion of the access guidewire 106 has completely escaped from the coupler 108 and the proximal end of the access guidewire 106 remains attached to the access-guidewire attachment point within the swivel-arm connector 194.) Notably, the introducer needle-withdrawing step is performed before the RICC-advancing step of advancing the catheter tube 112 over the access guidewire 106 and into the blood-vessel lumen.

FIG. 34 illustrates the RICC-advancing step of the method in accordance with some embodiments.

The RICC-advancing step includes advancing the catheter tube 112 of the RICC 102 over the access guidewire 106 and into the blood-vessel lumen, thereby inserting the RICC 102 into the blood-vessel lumen.

The access guidewire-withdrawing step includes withdrawing the access guidewire 106 leaving the catheter tube 112 in place in the blood-vessel lumen.

The maneuver guidewire-advancing step includes advancing a maneuver guidewire into the blood-vessel lumen by way of the primary lumen 128 of the RICC 102 and to a lower ⅓ of an SVC of a heart of the patient.

The other RICC-advancing step includes advancing the distal portion of the catheter tube 112 farther into the blood-vessel lumen over the maneuver guidewire to the lower ⅓ of the SVC of the heart of the patient.

The maneuver guidewire-withdrawing step includes withdrawing the maneuver guidewire leaving the catheter tube 112 in place in the lower 1/3 of the SVC.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

1. A rapidly insertable central catheter (“RICC”) insertion assembly, comprising:
a RICC including a primary lumen;
an introducer needle including a needle shaft having a longitudinal needle slot;
an access guidewire having a proximal portion disposed in the primary lumen of the RICC and a distal portion disposed in the needle shaft through the needle slot; and
a coupler coupling the RICC and the introducer needle together, the coupler including:
a coupler housing including a sealing-module cavity of a sealing module;
an elastomeric sealing-module insert of the sealing module inserted into the sealing-module cavity, the sealing-module insert configured to separately seal around a proximal portion of the introducer needle and the distal portion of the access guidewire when the sealing-module insert is compressed in the sealing-module cavity in one or more states of the RICC insertion assembly.

2. The RICC insertion assembly of claim 1, wherein the sealing-module insert includes an introducer-needle passageway and an access-guidewire passageway, the introducer-needle passageway configured to seal around the proximal portion of the introducer needle and the access-guidewire passageway configured to seal around the distal portion of the access guidewire when the sealing-module insert is compressed in the sealing-module cavity.

3. The RICC insertion assembly of claim 2, wherein the introducer-needle passageway includes an internal relief between proximal and distal portions of the introducer-needle passageway such that only proximal- and distal-end portions of the introducer-needle passageway are configured to seal around the proximal portion of the introducer needle when the sealing-module insert is compressed in the sealing-module cavity.

4. The RICC insertion assembly of claim 2, wherein the access-guidewire passageway includes an internal relief in at least a proximal portion of the access-guidewire passageway such that only a proximal-end portion of the access-guidewire passageway is configured to seal around the distal portion of the access guidewire when the sealing-module insert is compressed in the sealing-module cavity.

5. The RICC insertion assembly of claim 2, wherein a distal end of the access-guidewire passageway connects to a medial portion of the introducer-needle passageway, thereby allowing the distal portion of the access guidewire to be disposed in the needle shaft through the needle slot.

6. The RICC insertion assembly of claim 2, wherein the sealing-module insert includes a partial-length longitudinal slit between the access-guidewire passageway and the introducer-needle passageway configured to allow the access guidewire to escape from the access-guidewire passageway into the introducer-needle passageway after the introducer needle is withdrawn from the RICC insertion assembly through the introducer-needle passageway.

7. The RICC insertion assembly of claim 2, wherein the sealing-module insert includes a full-length longitudinal slit between the introducer-needle passageway and an exterior of the sealing-module insert configured to allow the access guidewire to escape from the introducer-needle passageway after the introducer needle is withdrawn from the RICC insertion assembly through the introducer-needle passageway.

8. The RICC insertion assembly of claim 7, wherein the full-length longitudinal slit in the sealing-module insert opens into a longitudinal coupler-housing slot of the coupler housing, the coupler-housing slot configured to allow the access guidewire to escape from the coupler housing after the introducer needle is withdrawn from the RICC insertion assembly through the introducer-needle passageway.

9. The RICC insertion assembly of claim 8, wherein the sealing-module insert includes an external relief between the sealing-module insert and the coupler housing coextensive with the full-length longitudinal slit in the sealing-module insert, the external relief configured to allow the access guidewire to escape from the introducer-needle passageway without binding the access guidewire in the full-length longitudinal slit.

10. The RICC insertion assembly of claim 1, wherein the sealing-module insert is radially compressed in the sealing-module cavity.

11. The RICC insertion assembly of claim 1, wherein the sealing-module insert is axially compressed in the sealing-module cavity by a follower proximal of the sealing-module insert, axial compression of the sealing-module insert in the sealing-module cavity, in turn, radially compressing the sealing-module insert in the sealing-module cavity.

12. The RICC insertion assembly of claim 11, wherein the follower is a distal portion of a needle hub of the introducer needle removably disposed in a needle-hub receptacle of the coupler housing, the needle hub having a proximal portion including a Luer connector for fluidly connecting a syringe to the introducer needle.

13. The RICC insertion assembly of claim 12, wherein the coupler includes a needle-hub lock configured to lock the needle hub in the needle-hub receptacle, a pair of lock buttons of the needle-hub lock distributed between opposite sides of the coupler configured to unlock the needle hub when the lock buttons are pressed into the coupler for withdrawal of the introducer needle from the coupler through the introducer-needle passageway.

14. The RICC insertion assembly of claim 12, the introducer needle further including a sheath over the needle shaft sealing the needle slot thereunder, the needle slot extending from a proximal portion of the needle shaft through a distal needle tip.

15. The RICC insertion assembly of claim 14, wherein the sealing module includes a blade disposed in the needle slot under a distal end of a sheath opening, the blade including a distal-facing blade edge configured to cut the sheath away from the needle shaft as the introducer needle is withdrawn from the coupler through the introducer-needle passageway, thereby allowing the access guidewire to escape from the needle shaft by way of the needle slot.

16. The RICC insertion assembly of claim 15, wherein the blade is overmolded into the sealing-module insert, thereby integrating the blade in the sealing-module insert.

17. The RICC insertion assembly of claim 15, wherein the blade extends from a blade holder of a blade module, the blade module disposed in a sealing module-insert cavity of the sealing-module insert.

18. The RICC insertion assembly of claim 1, wherein a proximal end of the access guidewire is coupled to a swivel arm swivelably coupled to the coupler housing, thereby enforcing a loop in the access guidewire with the distal portion of the access guidewire disposed in the needle shaft, over which loop the RICC is disposed in at least a ready-to-operate state of the RICC insertion assembly.

19. A rapidly insertable central catheter (“RICC”) insertion assembly, comprising:
a RICC including:
a catheter tube;
a catheter hub coupled to a proximal portion of the catheter tube;
one or more extension legs, each extension leg of the one-or-more extension legs coupled to the catheter hub by a distal portion thereof; and
one or more extension-leg connectors, each extension-leg connector of the one-or-more extension-leg connectors over a proximal portion of an extension leg of the one-or-more extension legs;
an introducer needle including:
a needle shaft including a longitudinal needle slot extending from a proximal portion of the needle shaft through a distal needle tip;
a sheath over the needle shaft, the sheath including a sheath opening in a proximal portion of the sheath; and
a needle hub over the proximal portion of the needle shaft and the proximal portion of the sheath;
a coupler coupling the RICC and the introducer needle together, the coupler including:
a coupler housing including:
a sealing-module cavity of a sealing module; and
a needle-hub receptacle proximal of the sealing-module cavity, the needle hub inserted into the needle-hub receptacle;
an elastomeric sealing-module insert of the sealing module inserted into the sealing-module cavity; and
an extension arm coupled to the coupler housing, the extension arm including an extension-arm connector connected to an extension-leg connector of the one-or-more extension-leg connectors; and
an access guidewire disposed in both the RICC and the introducer needle in at least a ready-to-operate state of the RICC insertion assembly, the access guidewire including:
a proximal end coupled to the extension-arm connector;
a proximal portion extending along a primary lumen of the RICC;
a distal portion extending along the primary lumen of the RICC, out a distal end of the RICC, into the sealing module over the needle hub, into the needle shaft through both the sheath opening and the needle slot, and along a needle lumen of the introducer needle; and
a distal end disposed in the needle lumen just proximal of the needle tip, thereby enforcing a loop in the access guidewire over which loop the RICC is disposed, the sealing-module insert separately sealing around both a proximal portion of the introducer needle and the distal portion of the access guidewire with the sealing-module insert compressed in the sealing-module cavity by the needle hub.

20-30. (canceled)
